(19) United States
(12) Patent Application Publication
(10) Pub. No.: US 2023/0090177 A1
(43) Pub. Date: Mar. 23, 2023

(54) METHODS FOR MAKING EXTRACELLULAR VESICLES AND USES THEREOF

(71) Applicant: GENENTECH, INC., South San Francisco, CA (US)

(72) Inventors: James Thomas Koerber, South San Francisco, CA (US); Yonglian Sun, South San Francisco, CA (US)

(73) Assignee: GENENTECH, INC., South San Francisco, CA (US)

(21) Appl. No.: 17/994,638

(22) Filed: Nov. 28, 2022

Related U.S. Application Data
(63) Continuation of application No. PCT/US2021/035072, filed on May 31, 2021.
(60) Provisional application No. 63/033,014, filed on Jun. 1, 2020.

(51) Int. Cl. G01N 33/531; G01N 33/566; C07K 16/18; C07K 16/10

(52) U.S. Cl. CPC G01N 33/531; G01N 33/566; C07K 16/18; C07K 16/1045; C07K 2317/24; C07K 2317/14

(57) ABSTRACT

The present disclosure relates to improved methods and compositions for making extracellular vesicles (EVs). The present disclosure also relates to novel EV-based ELISA assays and kits for performing such assays, as well as methods of producing antibodies to particular antigens using EVs comprising membrane-bound antigen.

### CROSS REFERENCE TO RELATED APPLICATIONS

This application is a continuation application of International Patent Application No. PCT/US2021/035072, filed May 31, 2021, which claims priority to U.S. Provisional Application No. 63/033,014, filed Jun. 1, 2020, the contents of each of which are incorporated by reference herein in their entireties and to each of which priority is claimed.

### INTRODUCTION

The present disclosure relates to improved methods and compositions for making extracellular vesicles (EVs). The present disclosure also relates to novel EV-based ELISA assays and kits for performing such assays, as well as methods of producing antibodies to particular antigens using EVs comprising a membrane-bound antigen of interest.

### BACKGROUND

Extracellular vesicles (EVs) are a heterogeneous group of cell-derived membranous structures that are enclosed by a lipid bilayer. EVs include exosomes, microvesicles, viral-like particles (VLPs) and apoptotic bodies (>1 μm) (Thèry et al., “Membrane vesicles as conveyors of immune responses,” Nat Rev Immunol. 2009; 9(8):581-93; Andaloussi et al., “Extracellular vesicles: biology and emerging therapeutic opportunities,” Nat Rev Drug Discov. 2013; 12(5):347-357). EVs can display membrane proteins in their native conformations on the EV surface in a highly concentrated manner. For example, membrane proteins can be present on the surface of EVs at concentrations of 10 to 100 times higher than on cell membranes.

Characteristics of a robust EV generating platform include one or more of the following: the ability to reproducibly incorporate both single and multi-pass membrane proteins; generation of sufficient EV yields (e.g., mg level); be easily transfected at a reasonable scale (e.g., about 1 L); and the ability to generate species-matched backgrounds. Prior methods of producing EVs are unable to meet all of these requirements.

### SUMMARY

The present disclosure relates to improved methods and compositions for making extracellular vesicles (EVs). The present disclosure also relates to novel EV-based ELISA assays and kits for performing such assays, as well as methods of producing antibodies to particular antigens using EVs comprising a membrane-bound antigen of interest.

In one aspect, the present disclosure provides methods for producing an antibody that specifically bind to a protein. In certain embodiments, the method includes (a) producing a plurality of EVs comprising a heterologous protein by (i) expressing the heterologous protein in a cell exposed to a vesicle factor, (ii) culturing the cell in a medium and (iii) isolating the plurality of EVs comprising the heterologous protein from the medium, wherein the vesicle factor is selected from the group consisting of Acyl.Hrs, ARRDC1, ARF6 and a combination thereof; (b) immunizing an animal by administering the plurality of EVs to the animal; and (c) isolating an antibody that binds to the heterologous protein from the animal.

Alternatively and/or additionally, a method for producing an antibody that specifically binds to a protein can include (a) producing a plurality of EVs comprising a heterologous protein by (i) expressing the heterologous protein in a cell, (ii) culturing the cell in a medium and (iii) isolating the plurality of EVs comprising the protein from the medium, wherein the cell is a non-adherent cell; (b) immunizing an animal by administering the plurality of EVs to the animal; and (c) isolating an antibody that binds to the heterologous protein from the animal. In certain embodiments, the method can further include expressing a vesicle factor, e.g., a heterologous vesicle factor, in the cell, e.g., MLGag, Acyl.Hrs, ARRDC1, ARF6 or a combination thereof.

In certain embodiments, the plurality of EVs are isolated from the medium by ultracentrifugation. In certain embodiments, the plurality of EVs is administered to the animal on week 0, week 2 and week 4. In certain embodiments, the method for producing an antibody further comprises administering an adjuvant to the animal concurrently with the EVs, e.g., a Ribi adjuvant. In certain embodiments, the method for producing an antibody further comprises administering a boost to the animal to enhance an immune response in the animal to the protein. In certain embodiments, the boost comprises the protein, a polynucleotide encoding the protein or a combination thereof.

The present disclosure further provides antibodies produced by the methods of the present disclosure. In certain embodiments, the antibody is a monoclonal antibody. In certain embodiments, the antibody is a human, humanized or chimeric antibody. The present disclosure further provides pharmaceutical compositions comprising the antibody or antigen-binding portion thereof and a pharmaceutically acceptable carrier. In certain embodiments, the pharmaceutical composition further includes an additional therapeutic agent. In certain embodiments, an antibody produced by the methods of the present disclosure, or pharmaceutical compositions thereof, can be used as a medicament, can be used in treating a disease and/or can be used in the manufacture of a medicament. In certain embodiments, the present disclosure provides methods of treating an individual having a disease, wherein the method includes administering to the individual an effective amount of an isolated antibody or antigen-binding portion thereof disclosed herein, or a pharmaceutical composition thereof. The present disclosure further provides isolated nucleic acids encoding an antibody or antigen-binding portion thereof disclosed herein, and host cells comprising the nucleic acid. The present disclosure also provides methods of producing an antibody by culturing the host cell under conditions suitable for expression of the antibody and, optionally, isolating the antibody from the host cell.

In a further aspect, the present disclosure provides a method for producing a plurality of EVs. In certain embodiments, the method includes (a) expressing a heterologous protein in a cell; (b) culturing the cell in a medium; and (c) isolating the plurality of EVs comprising the heterologous protein from the medium, wherein the cell is exposed to a vesicle factor selected from the group consisting of Acyl.Hrs, ARRDC1, ARF6 and a combination thereof, and/or wherein the cell is a non-adherent cell.

In certain embodiments, the heterologous protein is a membrane protein, e.g., a single-pass membrane protein or a multi-pass membrane protein. In certain embodiments, the membrane protein is a member of a protein complex. In certain embodiments, the membrane protein is not a transmembrane protein but is a member of a complex with a transmembrane protein. In certain embodiments, the non-adherent cell is a 293S cell or an Expi293™ cell.

In another aspect, the present disclosure provides methods for detecting an antibody. For example, but not by way of limitation, the method can include (a) incubating a sample with a capture reagent, wherein the capture reagent comprises a plurality of EVs comprising a membrane-bound antigen, and the antibody binds specifically to the membrane-bound antigen; and (b) contacting the antibody bound to the capture reagent with a detectable antibody to detect the bound antibody, wherein the detectable antibody binds specifically to the antibody. In certain embodiments, the plurality of EVs are generated by (i) expression of the membrane-bound antigen in a cell, (ii) culturing the cell in vitro in a medium to produce the plurality of EVs displaying the membrane-bound antigen and (iii) isolating the plurality of EVs displaying the membrane-bound antigen from the medium. In certain embodiments, the cell is exposed to a vesicle factor selected from the group consisting of Acyl.Hrs, ARRDC1, ARF6 and a combination thereof and/or the cell is a non-adherent cell. In certain embodiments, the method can further include (c) measuring the amount of the antibody detected in (b), wherein the amount is quantitated using a standard curve. In certain embodiments, the sample is a plasma, serum or urine sample. In certain embodiments, the capture antibody can be immobilized on a solid support, e.g., a microtiter plate. In certain embodiments, the detectable antibody is fluorescently labeled. In certain embodiments, the membrane-bound antigen is a membrane protein or a fragment thereof.

The present disclosure provides kits for detecting an antibody in a sample. In certain embodiments, a kit of the present disclosure includes (a) a capture reagent that comprises a plurality of EVs comprising membrane-bound antigen, wherein the antibody to be detected binds specifically to the membrane-bound antigen; and (b) a detectable antibody binds specifically to the antibody to be detected. In certain embodiments, the plurality of EVs are immobilized on a solid support. In certain embodiments, the solid support is a microtiter plate. In certain embodiments, the detectable antibody is fluorescently labeled. In certain embodiments, the antigen is a membrane protein or a fragment thereof.

In certain embodiments, the present disclosure further provides a method for sorting antibody-producing cells. In certain embodiments, the method includes incubating the antibody-producing cells with a plurality of EVs wherein the plurality of EVs comprise: (i) a first population of EVs comprising a membrane-bound antigen and a first detectable marker, wherein a subset of the antibody-producing cells bind specifically to the membrane-bound antigen; and (b) a second population of EVs lacking the membrane-bound antigen but comprising a second detectable marker distinguishable from the first marker. In certain embodiments, the method further includes sorting the antibody-producing cells based on their binding to either the first population of EVs or to a combination of the first population of EVs and the second population of EVs. In certain embodiments, the first population of EVs is generated by (i) expression of the membrane-bound antigen and the first detectable marker in a first cell, (ii) culturing the first cell in vitro in a medium to produce the plurality of EVs displaying the membrane-bound antigen and (iii) isolating the plurality of EVs displaying the membrane-bound antigen from the medium. In certain embodiments, the second population of EVs is generated by (i) expression of the second detectable marker in a second cell, (ii) culturing the second cell in vitro in a medium to produce the plurality of EVs comprising the second detectable marker and (iii) isolating the plurality of EVs from the medium. In certain embodiments, the cell is exposed to a vesicle factor selected from the group consisting of Acyl.Hrs, ARRDC1, ARF6 and a combination thereof. In certain embodiments, the first cell and/or second cell is a non-adherent cell.

### BRIEF DESCRIPTION OF THE DRAWINGS

FIG. 1. A schematic drawing showing the formation of extracellular vesicles (EVs).

FIGS. 2A-2B. Design principles for EV formation. (2A) General EV former and Acyl.Hrs possessing EV designs. (2B) MLGag and ARRDC1 possessing EV designs.

FIG. 3. A schematic drawing showing the workflow of producing EVs.

FIG. 4. Identification of vesicle factors that can produce EVs expressing MP-X using Western Blot.

FIG. 5. Dynamic Light Scattering (DLS) showing uniform vesicle sizes.

FIG. 6. Western Blot showed vesicle factors MLGag, Acryl.Hrs and murine ARRDC1 (mARRDC1) induced MP-X-expressing EV formation in murine cells.

FIGS. 7A-7B. EV production challenges: (7A) challenges in obtaining efficient EV purification; and (7B) challenges in obtaining sufficient yield.

FIGS. 8A-8C. Average yields of EVs (8A, 8B) and cell viability (8C) were measured on harvest day in Expi293™ cells and 293S cells, which were used for producing EVs expressing target proteins, having MLGag as the vesicle factor.

FIGS. 9A-9C. ELISA was performed to compare the expression levels of MP-7 (9A), MP-8 (9B), or MP-4 (9C) between the Expi293F cell produced EVs and 293S cell produced EVs.

FIGS. 10A-10C. (10A) Well-defined EV particles were generated. (10B, 10C) EV-based ELISA can detect FACS+ antibodies against single pass (10B) and multi-pass (10C) membrane proteins.

FIGS. 11A-11D. Cell lines were identified for screening Expi293F™ EV immunized rats and mice. (11A) A schematic drawing shows the animal immunization protocol. (11B) The binding of antiserum and prebleed to cells was shown by FACS. pAb from EV immunizations in rats bound 293 cells but not to RBA cells. pAb from EV immunizations in mice bound to RBA cells but not 3T3 cells. (11C, 11D) FACS using eGFP showed that both RBA (11C) and 3T3 (11D) were transfectable.

FIGS. 12A-12C. Cell lines were identified for screening Expi293™ EV immunized rabbits and llama/camels. (12A) A schematic drawing shows the animal immunization protocol. (12B) The binding of antiserum and prebleed to cells was shown by FACS. pAb from EV immunizations in rabbits did not bind RK13 cells. pAb from EV immunizations in llamas did bind 3T3 cells, but did not bind Dubca cells. (12C) FACS showed that both RK13 cells (left) and Dubca cells (right) were transfectable.

FIG. 13. Rat RBA cells can produce EVs, but the yields were low compared to 293S cells.

FIG. 14. Western blot confirmed the presence of MP-1 in whole cell lysate and EVs.

FIG. 15. Western blot confirmed that MP-2 was present in EVs and whole cell lysate.

FIG. 16. Western blot confirmed that MP-3 was present in ARF-6 possessing EVs produced from cells transfected with ARF-6, but not MLGag

FIG. 17. A schematic drawing showing that Gag capsid sterically blocks the incorporation of MPs with large intracellular domains (ICDs).

FIG. 18. A schematic drawing showing the working mechanisms of protein ELISA, EV-based ELISA, and FACS.

FIGS. 19A-19D. EV-based ELISA titer (19A) correlated well with FACS titer for MP-4 (19B). FACS (19C) and EV-based ELISA (19D) titers did not correlate well with protein ELISA titer.

FIGS. 20A-20B. Anti-MP-5 sera was collected from mice immunized with MP-5 using DNA immunization. EV-based ELISA titers (20A) and FACS titers (20B) are shown.

FIG. 21. EV-based ELISA correlated well with FACS for detecting anti-MP5 antibodies.

FIGS. 22A-22C. Quality Control (QC) analysis of the initial batch of MP-6 EVs. (22A) Western blot showed the presence of MP-6 in isolated EVs. (22B) Western blot showed the presence of the vesicle factors in isolated EVs. (22C) A quantitative Western blot using recombinant protein standard was used to quantify MP-6 in isolated EVs.

FIG. 23. Immunization protocols using MP-6 EVs.

FIGS. 24A-24D. (24A) Western blot showed the presence of anti-MP-6 and anti-Gag antibodies in the antiserum collected from rat immunized with EVs. (24B) FACS showed there was no significant non-specific binding of antiserum to transfection control cells. (24C, 24D) FACS showed the binding to MP-6 expressing cells in antiserum collected before DNA/protein boost (24C) and after DNA/protein boost (24D).

FIGS. 25A-25B. Purified primary antibodies (25A) and serum (25B) showed similar FACS results.

FIG. 26. DNA boost selectively increased anti-MP-6 titer from EV immunized rats.

FIGS. 27A-27C. Mouse anti-MP-7 primary antibodies were generated from knockout mice immunized with MP-7 expressing EVs, and screened by FACS. Anti-MP-7 antibodies were detected in serum collected before last boost (27A) and after the last boost (27B). Serum did not bind 3T3 control cells (27C).

FIG. 28. Mouse anti-MP-7 hybridomas were screened by FACS.

FIG. 29. Mouse anti-MP-7 hybridomas were screened by FACS.

FIG. 30. Screening mouse anti-MP-7 mAbs using FACS on primary cells.

FIGS. 31A-31B. Rats were immunized with EVs comprising membrane-bound MP-1 or MP-1 DNA, and with protein or DNA boost. Antiserum collected from the rats before boost (31A) and after boost (31B) were screened by FACS.

FIG. 32. Rats anti-MP-1 hybridomas were screened by FACS.

FIG. 33. Rats were immunized with only protein, DNA, or EVs comprising membrane-bound MP-8. The number of ELISA-positive and FACS-positive antibodies discovered from each group are shown.

FIG. 34. Rats and rabbits were immunized with EVs comprising MP-9 and MP-9 DNA. Staining of rat and rabbit IgG+ B cells with GFP-labeled MP-9 EVs and RFP-labeled empty EVs are shown.

FIG. 35. Rats and rabbits were immunized with EVs comprising MP-10 or MP-11. Staining of rabbit IgG+ B cells with RFP-labeled MP EVs and GFP-labeled empty EVs are shown.

FIG. 36A. Co-expression of MP-14, MP-15, MP-16 and MP-17 (Co-receptor “B”) and MP-12 and MP-13 (Receptor “A”) is required for surface expression.

FIG. 36B. Co-expression of MP-14, MP-15, MP-16 and MP-17 (Co-receptor “B”) and MP-12 and MP-13 (Receptor “A”) results in EV incorporation.

### DETAILED DESCRIPTION

The present disclosure relates to improved methods and compositions for making extracellular vesicles (EVs). The present disclosure also relates to novel EV-based ELISA assays and kits for performing such assays, as well as methods of producing antibodies to particular antigens using EVs comprising a membrane-bound antigen of interest, e.g., a membrane protein. The present disclosure is based, in part, on the discovery that by adopting certain purification steps, vesicle factors, and/or EV producing cell lines, it is possible to achieve rapid and high yield generation of EVs. It is also based, in part, on the discovery that immunizing animals with antigen-presenting EVs allows for the development of functional antibodies against challenging membrane protein antigens and complexes. Non-limiting embodiments of the instant disclosure are described herein.

For purposes of clarity of disclosure and not by way of limitation, the detailed description is divided into the following subsections:

I. Definitions;

II. Methods of Making EVs;

III. EV-based ELISA Assays and Kits;

IV. Methods of Producing Antibodies Using EVs;

V. Methods and Compositions for Diagnostics and Detection;

VI. Pharmaceutical Compositions;

VII. Therapeutic Methods and Routes of Administration;

VIII. Articles of Manufacture; and

IX. Exemplary Embodiments.

### I. Definitions

The terms used in the instant disclosure generally have their ordinary meanings in the art, within the context of the present disclosure and in the specific context where each term is used. Certain terms are discussed below, or elsewhere in the instant disclosure, to provide additional guidance to the practitioner in describing the compositions and methods of the instant disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. In certain embodiments, the individual or subject is a human.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “biological sample” refers to a sample of biological material obtained from a subject, including a biological fluid, e.g., blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid, bronchoalveolar fluid, biliary fluid and combinations thereof.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

As used herein, the term “antibody fragment” refers to a molecule other than an intact antibody that comprises a portion of an intact antibody that binds the antigen to which the intact antibody binds. Examples of antibody fragments include but are not limited to Fv, Fab, Fab′ Fab′-SH, F(ab′)₂; diabodies; linear antibodies; single-chain antibody molecules (e.g., scFv); and multispecific antibodies formed from antibody fragments.

As used herein, the term “chimeric antibody” refers to an antibody in which a portion of the heavy and/or light chain is derived from a particular source or species, while the remainder of the heavy and/or light chain is derived from a different source or species.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present disclosure can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody can be present in a pharmaceutical composition.

The “class” of an antibody refers to the type of constant domain or constant region possessed by its heavy chain. There are five major classes of antibodies: IgA, IgD, IgE, IgG, and IgM, and several of these can be further divided into subclasses (isotypes), e.g., IgG₁, IgG₂, IgG₃, IgG₄, IgA₁, and IgA₂. In certain embodiments, the antibody is of the IgG₁ isotype. In certain embodiments, the antibody is of the IgG₁ isotype with the P329G, L234A and L235A mutation to reduce Fc-region effector function. In other embodiments, the antibody is of the IgG₂ isotype. In certain embodiments, the antibody is of the IgG₄ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody can be assigned to one of two types, called kappa (κ) and lambda (λ), based on the amino acid sequence of its constant domain.

As used herein, the term “Framework” or “FR” refers to variable domain residues other than hypervariable region (CDR) residues. The FR of a variable domain generally consists of four FR domains: FR1, FR2, FR3, and FR4. Accordingly, the CDR and FR sequences generally appear in the following sequence in VH (or VL): FR1-H1 (L1)-FR2-H2(L2)-FR3-H3 (L3)-FR4.

As used herein, the terms “full length antibody,” “intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In certain embodiments, for the VL, the subgroup is subgroup kappa I as in Kabat et al., supra. In certain embodiments, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally can comprise at least a portion of an antibody constant region derived from a human antibody. A “humanized form” of an antibody, e.g., a non-human antibody, refers to an antibody that has undergone humanization.

The term “hypervariable region” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Unless otherwise indicated, CDR residues and other residues in the variable domain (e.g., FR residues) are numbered herein according to Kabat et al., supra. Generally, antibodies comprise six CDRs: three in the VH (H1, H2, H3), and three in the VL (L1, L2, L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including CDR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

An “immunoconjugate” is an antibody conjugated to one or more heterologous molecule(s), including but not limited to a cytotoxic agent.

The term “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the present disclosure in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see, e.g., Stadler et al., Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the terms “antigen” and “immunogen” are used interchangeably herein to refer to a molecule or substance which induces an immune response (preferably an antibody response) in an animal immunized therewith. The antigen can be a protein, peptide, carbohydrate, nucleic acid, lipid, hapten or other naturally occurring or synthetic compound. In certain embodiments, the antigen is a protein. In certain embodiments, the antigen is a membrane protein or a fragment thereof. In certain embodiments, the antigen is a single-pass or a multi-pass membrane protein or a fragment thereof.

As used herein, the term “heterologous protein” refers to a protein that is expressed in a cell by introducing a polynucleotide into the cell that encodes the heterologous protein. In certain embodiments, the heterologous protein is not native to the cell. In certain embodiments, the heterologous protein is a protein that is native to the cell but is overexpressed because of the introduction of a polynucleotide encoding the heterologous protein into the cell.

The terms “membrane-bound antigen” and “membrane antigen,” as used interchangeably herein, refer to an antigen that is bound to a membrane directly or indirectly.

The terms “membrane-bound protein” and “membrane protein,” as used interchangeably herein, refer to a protein that is bound to a membrane directly or indirectly. Non-limiting examples of membrane-bound proteins include integral, lipid-anchored and peripheral proteins. In certain embodiments, the membrane protein is a transmembrane protein, e.g., a single-pass or a multi-pass membrane protein or a fragment thereof. In certain embodiments, the membrane protein is not a transmembrane protein but is a protein that is part of a complex with a transmembrane protein (e.g., a cofactor). As used in the examples herein, the acronym “MP” refers to a membrane protein.

As used herein, the term “transmembrane antigen” refers to an antigen that spans across a membrane at least once. Non-limiting examples of transmembrane antigens include single pass antigens, e.g., antigens that span a membrane once, lipid-anchored proteins, or multi-pass antigens, e.g., proteins that span a membrane at least twice.

As used herein, the term “transmembrane protein” refers to a protein that spans across a membrane at least once. Non-limiting examples of transmembrane proteins include single pass proteins, e.g., proteins that span a membrane once, lipid-anchored proteins, or multi-pass proteins, e.g., proteins that span a membrane at least twice. In certain embodiments, the transmembrane protein is a single-pass or a multi-pass transmembrane protein or a fragment thereof. In certain embodiments, the transmembrane protein is a multi-pass transmembrane protein or a fragment thereof.

As used herein, the term “immunizing” refers to the step or steps of administering one or more antigens to an animal so that antibodies can be raised in the animal. Generally, immunizing comprises injecting the antigen or antigens into the animal. Immunization can involve one or more administrations of the antigen or antigens. In certain embodiments, the antigen is administered to the animal though a plurality EVs that express the antigen.

As used herein, the term “polyclonal antibodies” or “polyclonal antisera” refer to immune serum containing a mixture of antibodies specific for one (monovalent or specific antisera) or more (polyvalent antisera) antigens which can be prepared from the blood of animals immunized with the antigen or antigens.

As used herein, the term “adjuvant” refers to nonspecific stimulant of the immune response. The adjuvant can be the form of a composition comprising either or both of the following components (a) a substance designed to form a deposit protecting the antigen(s) from rapid catabolism (e.g., mineral oil, alum, aluminum hydroxide, liposome or surfactant [e.g., pluronic polyol]) and (b) a substance that nonspecifically stimulates the immune response of the immunized host animal (e.g., by increasing lymphokine levels therein). Non-limiting examples of molecules for increasing lymphokine levels include lipopolysaccharide (LPS) or a Lipid A portion thereof, Bordetella pertussis, pertussis toxin, Mycobacterium tuberculosis, and muramyl dipeptide (MDP). Non-limiting examples of adjuvants include Freund's adjuvant (optionally comprising killed M. tuberculosis to form Freund's complete adjuvant (FCA)), aluminum hydroxide adjuvant, Ribi adjuvants, Titermax adjuvants, specol adjuvants, aluminum salt adjuvants, and monophosphoryl Lipid A-synthetic trehalose dicorynomylcolate (MPL-TDM).

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the present disclosure are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (CDRs). (See, e.g., Kindt et al. Kuby Immunology, 6^{th} ed., W.H. Freeman and Co., page 91 (2007).) A single VH or VL domain can be sufficient to confer antigen-binding specificity. Furthermore, antibodies that bind a particular antigen can be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

As used herein, the term “screening” refers to subjecting one or more monoclonal antibodies (e.g., purified antibody and/or hybridoma culture supernatant comprising the antibody) to one or more assays which determine qualitatively and/or quantitatively the ability of an antibody to bind to an antigen of interest.

As used herein, a “marker” refers to compositions that allow for direct or indirect detection. Markers include, but are not limited to, fluorescent compositions, chromogenic labels, electron dense labels, chemiluminescent labeles and radioactive labels. For example, but not by limitation, specific markers are green fluorescent protein (“GFP”), mCherry, dtTomato, or other fluorescent proteins known in the art (e.g., Shaner et al., A Guide to Choosing Fluorescent Proteins, Nature Methods 2(12) 905-909 (December 2005) incorporated by reference herein, ³²P, ¹⁴C, ¹²⁵I, ³H and ¹³¹I, fluorogens (such as Rare Earth Chelate or lucifer yellow and its derivatives), Rhodamine (rhodamine) and its derivatives, dansyl, umbelliferone, luciferase (such as firefly luciferase and bacterial fluorescence plain enzyme) (U.S. Pat. No. 4,737,456), fluorescein, 2,3-dihydros phthalazine diketone, as well as enzymes producing detectable signals, e.g., horseradish peroxidase (HRP), alkaline phosphorus sour enzyme, beta galactosidase, glucoamylase, lysozyme, carbohydrate oxidase (such as glucose oxidase, galactose oxidase and glucose-6-phosphate dehydrogenase (G6PD)), and heterocyclic oxidases (such as uricase and xanthine oxidase).

As used herein, an “adherent cell” refers to a cell that requires attachment to a surface for growth.

As used herein, a “non-adherent cell” refers to a cell that is cultured in suspension. In certain embodiments, non-adherent cells are cells that do not require attachment to a surface for growth.

### II. Methods of Making EVs

In one aspect, the present disclosure relates to improved methods of making EVs. It is based, in part, on the discovery that by adopting certain purification steps, vesicle factors, and/or EV producing cell lines, it is possible to achieve rapid and high yield generation of EVs.

In certain non-limiting embodiments, a method for producing EVs comprises: (a) expressing a protein of interest, e.g., a heterologous protein of interest, in a cell exposed to a vesicle factor; (b) culturing the cell in vitro in a medium to produce a plurality of EVs; and (c) isolating the plurality of EVs from the medium. In certain embodiments, exposing the cell to a vesicle factor comprises expressing the vesicle factor in the cell.

In certain embodiments, expressing a protein of interest in a cell includes introducing at least one polynucleotide that encodes the protein of interest into the cell. For example, but not by way of limitation, a method for producing EVs comprises: (a) introducing a polynucleotide encoding a protein of interest, e.g., a heterologous protein of interest, in a cell exposed to a vesicle factor; (b) culturing the cell in vitro in a medium to produce a plurality of EVs; and (c) isolating the plurality of EVs from the medium. In certain embodiments, the cell can be transfected with the polynucleotide to express the protein of interest in a cell.

In certain embodiments, exposing a cell to a vesicle factor can include expressing the vesicle factor in the cell, e.g., in the same cell that expresses the protein of interest. For example, but not by way of limitation, a polynucleotide encoding the vesicle factor can be introduced into the cell. In certain embodiments, the vesicle factor can be encoded by the same polynucleotide that encodes the protein of interest. Alternatively, the protein of interest and the vesicle factor can be encoded by two different polynucleotides. For example, but not by way of limitation, expressing a protein of interest in a cell exposed to a vesicle factor includes introducing a first polynucleotide encoding the vesicle factor and a second polynucleotide encoding the protein of interest into the cell.

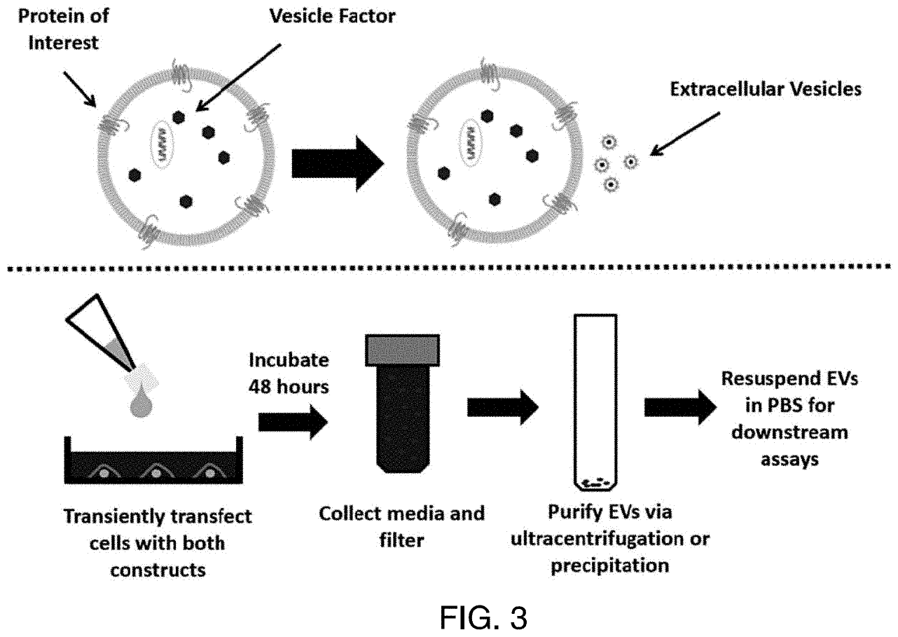

In certain non-limiting embodiments, a method for producing EVs comprises: (a) providing (i) a polynucleotide encoding a vesicle factor and a protein of interest and/or (ii) a first polynucleotide encoding a vesicle factor and a second polynucleotide encoding a protein of interest; (b) transfecting a cell with the polynucleotide(s), e.g., the polynucleotide or the first and second polynucleotides; (c) culturing the cell in vitro in a medium to produce a plurality of EVs; and (d) isolating the plurality of EVs from the medium. In certain embodiments, the first polynucleotide and second polynucleotide are provided on a single nucleic acid. An exemplary EV generation workflow is shown in FIG. 3.

In certain embodiments, exposing a cell to a vesicle factor can include expressing a vesicle factor in a cell distinct from the cell that expresses the protein of interest. For example, but not by way of limitation, a method of producing EVs can include expressing the protein of interest within a cell, e.g., a first cell. In certain embodiments, the protein of interest can be expressed in the cell by introducing a polynucleotide that encodes the protein of interest in the cell. In certain embodiments, the method can further include expressing a vesicle factor within a different cell, e.g., a second cell, that is co-cultured with the cell that expresses the protein of interest, e.g., the first cell. In certain embodiments, the method includes exposing the cell expressing the protein of interest, e.g., the first cell, to the vesicle factor expressed by the other cell, e.g., the second cell, to produce EVs that display the protein of interest.

In certain non-limiting embodiments, a method for producing EVs can include expressing the protein of interest in a cell in the absence of a vesicle factor. In certain embodiments, the method comprises: (a) expressing a heterologous protein of interest in a cell; (b) culturing the cell in vitro in a medium to produce a plurality of EVs; and (c) isolating the plurality of EVs from the medium, where the cell is a non-adherent cell. In certain non-limiting embodiments, a method of the present disclosure comprises: (a) providing a polynucleotide encoding a protein of interest; (b) transfecting a cell with the polynucleotide; (c) culturing the cell in vitro in a medium to produce a plurality of EVs; and (d) isolating the plurality of EVs from the medium.

In certain embodiments, a method of producing EVs can include expressing two or more proteins that form a protein complex in a cell, e.g., expressing two or more heterologous proteins that form a complex in a cell. For example, but not by way of limitation, a method of the present disclosure can include expressing two or more proteins of a protein complex, e.g., three or more proteins, four or more proteins, five or more proteins, six or more proteins, seven or more proteins, eight or more proteins or nine or more proteins of a protein complex, in a cell. In certain embodiments, one or more proteins of the protein complex expressed in the cell can be a transmembrane protein. In certain embodiments, one or more proteins of the protein complex expressed in the cell is not a transmembrane protein. In certain embodiments, one or more proteins of the protein complex expressed in the cell can be a peripheral membrane protein, e.g., a protein that is associated with a transmembrane protein. In certain embodiments, the two or more proteins can be expressed in the cell by introducing a polynucleotide that encodes the proteins or by introducing two or more polynucleotides that encode the proteins. In certain embodiments, the method can further include exposing the cell to a vesicle factor, e.g., by expressing a vesicle factor within the cell, e.g., to produce EVs that display the complex of the two or more proteins. Alternatively and/or additionally, the protein(s) of interest can be expressed in a cell that produces large number of EVs, e.g., non-adherent cells, in the absence of a vesicle factor.

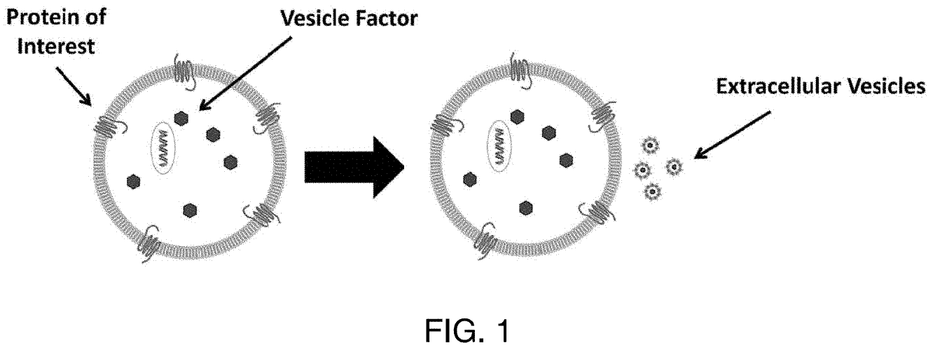

In certain embodiments, the vesicle factors are candidate proteins that can boost natural vesicle pathways or directly induce vesicle formation (FIG. 1). Non-limiting exemplary vesicle factors and their working mechanisms are shown in Table 1. In certain embodiments, a cell can be genetically-modified to express one or more of the vesicle factors disclosed herein. In certain embodiments, the vesicle factor is selected from the group consisting of MLGag, Acyl.Hrs, ARRDC1, RhoA, e.g., RhoA.F30L, ARF6, e.g., ARF6.Q67L, and a combination thereof. In certain embodiments, the vesicle factor is selected from the group consisting of MLGag, Acyl.Hrs, ARRDC1 and ARF6, e.g., ARF6.Q67L, and a combination thereof. In certain embodiments, the vesicle factor is selected from the group consisting of Acyl.Hrs, ARRDC1 and ARF6, e.g., ARF6.Q67L and a combination thereof.

TABLE 1
Vesicle factors and their working mechanisms.

| Mechanism | Protein |
|---|---|
| Self-assembling VLP | MLGag |
| Self-assembling VLP | ARRDC1, Acyl.Hrs |
| Enhance endogenous pathways | RhoA.F30L, ARF6.Q67L, VPS4a, |
| (e.g. exosome, tumor) | HAS3, CD9, CD63, CD81 |
| Apoptotic bodies | Constitutively active ROCK1 |

In certain embodiments, the vesicle factor is a Gag protein, e.g., a chimeric Gag protein. HIV viral Gag protein contains a peptide that binds and recruits the Tsg101/ESCRT complex to the membrane to facilitate viral budding (Pornillos et al., “HIV Gag mimics the Tsg101-recruiting activity of the human Hrs protein,” J Cell Biol. 2003; 162(3): 425-434). Introducing the cDNA encoding for Gag alone into human cells has been shown to generate vesicles (see, e.g., Qiu et al. J. Virol. 1999, 73(11):9145-9152; and Megede et al. J. Virol. 2000, 74(6):2628-2635). However, wildtype HIV Gag does not bud efficiently in non-human cells. It has been shown that chimeric Gag protein can induce EV production in both human and murine cells (Hammarstedt et al., “Passive and active inclusion of host proteins in human immunodeficiency virus Type 1 Gag particles during budding at the plasma membrane,” J Virol. 2004; 78(11):5686-97; Chen et al., “Efficient assembly of an HIV-1/MLV Gag-chimeric virus in murine cells,” Proc Natl Acad Sci USA. 2001; 98(26):15239-44). An exemplary chimeric Gag (MLGag) is disclosed in Chen et al., “Efficient assembly of an HIV-1/MLV Gag-chimeric virus in murine cells,” Proc Natl Acad Sci USA. 2001; 98(26):15239-44, and the contents of which is incorporated by reference in its entirety. In certain embodiments, the chimeric Gag protein comprises a portion of HIV Gag and a portion of Gag from a different retrovirus. For example, but not by way of limitation, the chimeric Gag comprises an HIV Gag, where a region of the HIV Gag known to direct its localization is replaced with functionally homologous regions from Moloney murine leukemia virus (MLV), a murine retrovirus. In certain embodiments, the replaced region of the HIV Gag is a matrix domain (MA) to generate a chimeric Gag referred to herein as MLGag. In certain embodiments, chimeric and full-length Gag proteins can be generated from endogenous retroviruses (ERVs) sequences derived from any species, e.g., as described in Stocking et al. Cell Mol. Life Sci. 65(21):3383-3398 (2008), the contents of which is incorporated by reference in its entirety. In certain embodiments, the vesicle factor is MLGag.

In certain embodiments, the vesicle factor is an arrestin domain-containing protein 1 (ARRDC1). In certain embodiments, the vesicle factor is a murine ARRDC1 (mARRDC1). In certain embodiments, the vesicle factor is a human ARRDC1 (hARRDC1). ARRDC1 is a tetrapeptide PSAP motif of an accessory protein and is a host protein that induces EV formation. It has been shown that overexpression of ARRDC1 results in enhanced microvesicle (MV) formation. Such effect is mediated by the recruitment of Tsg101 via PSAP/PTAP peptide. Overexpression of ATPase VPS4a results in further enhancement in MV formation (Nabhan et al., “Formation and release of arrestin domain-containing protein 1-mediated microvesicles (ARMMs) at plasma membrane by recruitment of TSG101 protein,” Proc Natl Acad Sci USA. 2012; 109(11):4146-51).

In certain embodiments, the vesicle factor is ADP ribosylation factor-6 (ARF6). It has been shown that ARF6 is a Rho GTPase that drives microvesicle formation in tumor cells in an ERK-dependent manner (Muralidharan-Chari et al., “ARF6-regulated shedding of tumor cell-derived plasma membrane microvesicles,” Curr Biol. 2009; 19(22):1875-85). In certain embodiments, the vesicle factor is a constitutively active form of ARF6. For example, but not by way of limitation, the constitutively active form of ARF6 is ARF6.Q67L (see, e.g., Peters et al. J. Cell Biol 128(6):1003-1017 (1995), which is incorporated by reference in its entirety herein).

In certain embodiments, the vesicle factor is a mutant RhoA/ROCK1 that can also drive microvesicle formation in tumor cells (Li et al., “RhoA triggers a specific signaling pathway that generates transforming microvesicles in cancer cells,” Oncogene. 2012; 31(45):4740-9). In certain embodiments, the vesicle factor is a constitutively active form of RhoA. For example, but not by way of limitation, the constitutively active form of RhoA is RhoA.F30L (see, e.g., Lin et al. JBC 274(33):23633-23641 (1999), which is incorporated by reference in its entirety herein).

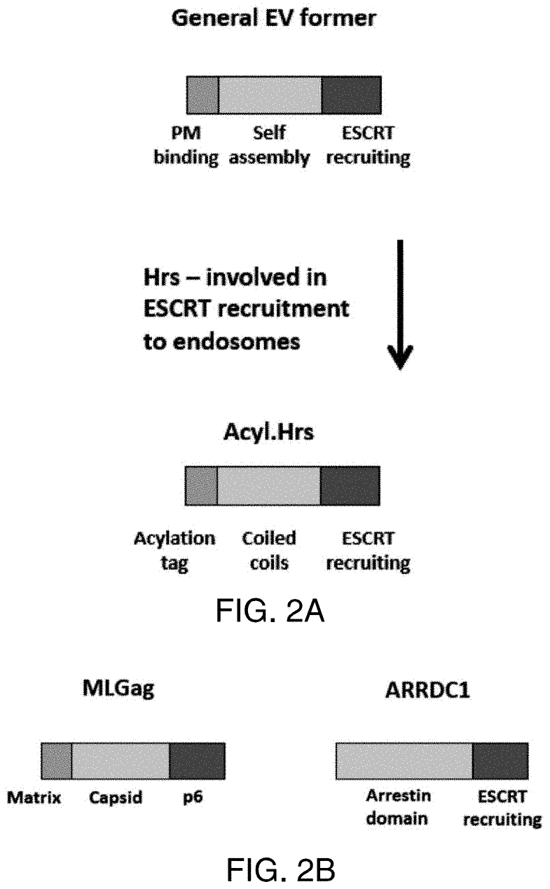

In certain embodiments, the vesicle factor comprises a plasma membrane (PM) binding domain, a self-assembly domain, and an endosomal sorting complex required for transport (ESCRT) recruiting domain (FIG. 2A). The design principle for EV formation is to enable rapid generation of new EV factors/cargo. It has been shown that PM targeting and high order oligomerization drives EV incorporation (Fang et al., “Higher-Order Oligomerization Targets Plasma Membrane Proteins and HIV Gag to Exosomes,” PLoS Biol. 2007 June; 5(6):e158). In certain embodiments, the vesicle factor is Acyl.Hrs that comprises a PM binding domain of acylation tag and the C-terminal domain of hepatocyte growth factor-regulated tyrosine kinase substrate (Hrs) that consists of a self-assembly domain of coiled coils, and an ESCRT recruiting domain (FIG. 2A). In certain embodiments, the vesicle factor is MLGag that comprises a PM binding domain of Matrix, a self-assembly domain of capsid, and an ESCRT recruiting domain of p6 (FIG. 2B). In certain embodiments, the vesicle factor comprises a self-assembly domain and an ESCRT recruiting domain. In certain embodiments, the vesicle factor is ARRDC1 that comprises a self-assembly domain of arrestin domain, and an ESCRT recruiting domain (FIG. 2B). Additional vesicle factors can be identified by any method known in the art. For example, but not by way of limitation, a screen of a cDNA library of all proteins, e.g., human proteins, can be performed to identify a single gene or a combination of genes that increases production of EVs. Alternatively or additionally, a CRISPR or RNAi screen can be performed to identify a single gene or a combination of genes that inhibits production of EVs.

In certain embodiments, the EVs produced by the methods disclosed herein comprise the vesicle factor and/or protein of interest. For example, but not by way of limitation, the vesicle factor is incorporated into the EVs, e.g., resides in the interior of the produced EVs. In certain embodiments, the protein of interest is displayed on the surface of the EV, e.g., the protein of interest is a protein that spans across the membrane one or more times or is a protein that is associated with a protein that spans across the membrane. In certain embodiments, EVs produced by the disclosed method comprise a vesicle factor, e.g., MLGag, Acyl.Hrs, ARRDC1 and/or ARF6, e.g., ARF6.Q67L, and the protein of interest. For example, but not by way of limitation, an EV produced by a method disclosed herein comprises ARF6, e.g., ARF6.Q67L, and a protein of interest. In certain embodiments, an EV produced by a method disclosed herein comprises MLGag and a protein of interest. In certain embodiments, an EV produced by a method disclosed herein comprises Acyl.Hrs and a protein of interest. In certain embodiments, an EV produced by a method disclosed herein comprises ARRDC1 and a protein of interest.

In certain embodiments, the vesicle factor is one or more of Acyl.Hrs, ARRDC1 and ARF6. In certain embodiments, the use of one or more of Acyl.Hrs, ARRDC1 and ARF6 is advantageous, as the use of Gag as the vesicle factor has been associated with the generation of anti-Gag antibodies. The production of anti-Gag antibodies can impact the ability of the immune system of an immunized animal to produce antibodies against the protein of interest potentially resulting in reduced titers of antibodies against the protein of interest.

As shown in Table 2, Acyl.Hrs, ARRDC1 and ARF6, produced similar amounts of EVs as MLGag. The results shown in Table 2 are surprising as Gag has evolved in the context of budding viruses from a cell surface and thus is expected to efficiently produce EVs, while the other vesicles factors, e.g., Acyl.Hrs, ARRDC1 and ARF6, did not evolve in such contexts, yet still resulted in high yields of EVs.

In certain embodiments, the vesicle factor, e.g., Acyl.Hrs, ARRDC1 and/or ARF6, can be from the same species that is to be immunized with EVs produced by expression of the vesicle factor. The use of a vesicle factor that is from the same species that is to be immunized with EVs produced by expression of the vesicle factor is advantageous as it can reduce the risk of an immune response to the vesicle factor rather than the protein of interest in the immunized animal. For example, but not by way of limitation, if a mouse is to be immunized to generate antibodies against a protein of interest, the vesicle factor, e.g., Acyl.Hrs, ARRDC1 and/or ARF6, can be from mouse, e.g., mouse Acyl.Hrs, mouse ARRDC1 and/or mouse ARF6. In certain embodiments, if a rat is to be immunized to generate antibodies against a protein of interest, the vesicle factor, e.g., Acyl.Hrs, ARRDC1 and/or ARF6, can be from rat, e.g., rat Acyl.Hrs, rat ARRDC1 and/or rat ARF6. In certain embodiments, if a rabbit is to be immunized to generate antibodies against a protein of interest, the vesicle factor, e.g., Acyl.Hrs, ARRDC1 and/or ARF6, can be from rabbit, e.g., rabbit Acyl.Hrs, rabbit ARRDC1 and/or rabbit ARF6. In certain embodiments, if a llama is to be immunized to generate antibodies against a protein of interest, the vesicle factor, e.g., Acyl.Hrs, ARRDC1 and/or ARF6, can be from llama, e.g., llama Acyl.Hrs, llama ARRDC1 and/or llama ARF6. In certain embodiments, if a human is to be immunized to generate antibodies against a protein of interest, the vesicle factor, e.g., Acyl.Hrs, ARRDC1 and/or ARF6, can be from human, e.g., human Acyl.Hrs, human ARRDC1 and/or human ARF6.

In certain embodiments, cells are modified to express a vesicle factor. For example, but not by way of limitation, a polynucleotide encoding a vesicle factor is introduced into the cell to express the vesicle factor. In certain embodiments, the cell is transfected with a polynucleotide encoding the vesicle factor to express the vesicle factor in the cell.

In certain embodiments, the cells are cultured under conditions suitable for expression of the vesicle factor. In certain embodiments, the cells are cultured under conditions suitable for production of EVs. For example, but not by way of limitation, the cells are cultured in a cell culture medium for expression of the vesicle factor and/or production of EVs.

In certain embodiments, the cells expressing a vesicle factor are incubated for a suitable time to produce EVs. In certain embodiments, the cells expressing a vesicle factor are incubated for about 12 hours to about 72 hours to produce EVs. In certain embodiments, the cells expressing a vesicle factor are incubated for about 24 hours to about 64 hours to produce EVs. In certain embodiments, the cells expressing a vesicle factor are incubated for about 48 hours to produce EVs.

In certain embodiments, the EVs produced by incubation of the cells expressing a vesicle factor are subsequently purified. In certain embodiments, the EVs are purified from the cell culture medium. In certain embodiments, purification of EVs takes from about 30 minutes to about 24 hours to complete. In certain embodiments, purification of EVs takes from about 30 minutes to about 12 hours to complete. In certain embodiments, purification of EVs takes from about 30 minutes to about 5 hours to complete. In certain embodiments, purification of EVs takes from about 30 minutes to about 4 hours to complete, e.g., about 1 hour to about 4 hours to complete. In certain embodiments, purification of EVs takes about 3 hours to complete. In certain embodiments, the EVs are isolated from the cell culture medium using ultracentrifugation.

In certain embodiments, the methods described herein for producing EVs using a vesicle factor are capable of producing about 0.5 mg or more, e.g., 0.5-1.0 mg; about 1.0 mg or more, e.g., 1.0-1.5 mg; about 1.5 mg or more, e.g., 1.5-2.0 mg; about 2.0 mg or more, e.g., 2.0-3.0 mg; about 2.5 mg or more, e.g., 2.5-3.0 mg; about 3.0 mg or more, e.g., 3.0-4.0 mg; about 3.5 mg or more, e.g., 3.5-4.0 mg; about 4.0 mg or more, e.g., 4.0-5.0 mg; about 4.5 mg or more, e.g., 4.5-5.0 mg; about 5.0 mg or more, e.g., 5.0-6.0 mg; or about 5.5 mg of more, e.g., 5.5-6.0 mg, of purified EVs. In certain embodiments, the methods described herein for producing EVs using a vesicle factor are capable of producing about 3.0 mg or more, e.g., 3.0-5.0 mg, of purified EVs. In certain embodiments, the methods described herein for producing EVs using a vesicle factor are capable of producing the aforementioned amounts of EVs within about 24-72 hours of culturing the cells expressing the heterologous protein. In certain embodiments, the methods described herein for producing EVs using a vesicle factor are capable of producing the aforementioned amounts of EVs within about 24-48 hours of culturing the cells expressing the heterologous protein. In certain embodiments, the methods described herein for producing EVs using a vesicle factor are capable of producing the aforementioned amounts of EVs within about 48-72 hours of culturing the cells expressing the heterologous protein.

In certain embodiments, the cell for use in the methods disclosed herein for EV production is a mammalian cell. In certain embodiments, the cell is a human cell. In certain embodiments, the cell is a genetically modified human cell. In certain embodiments, the cell is an adherent cell. For example, but not by way of limitation, the cell can be a HEK293 cell that grows adherently. HEK293 is a cell line derived from human embryonic kidney cells grown in tissue culture. In certain embodiments, the cell is a CHO cell. For example, but not by way of limitation, the cell is an ExpiCHO™ cell (ThermoFisher Scientific).

In certain embodiments, the cell used in the methods disclosed herein for EV production is not an adherent cell. In certain embodiments, the cell used in the methods disclosed herein for EV production is a non-adherent cell, e.g., a cell that grows in suspension. In certain embodiments, the non-adherent cell is a HEK293 cell that has been adapted for suspension culture. For example, but not by way of limitation, the cell is a 293 S cell, which is a HEK293 cell adapted for suspension culture. In certain embodiments, the cell is an Expi293F™ cell (ThermoFisher Scientific), which is derived from HEK293 cells and is maintained in suspension culture. As shown in Example 2, non-adherent cells, e.g., 293S cells and Expi293F™ cells, produced the highest yields of EVs compared to adherent cells, e.g., HEK293 cells.

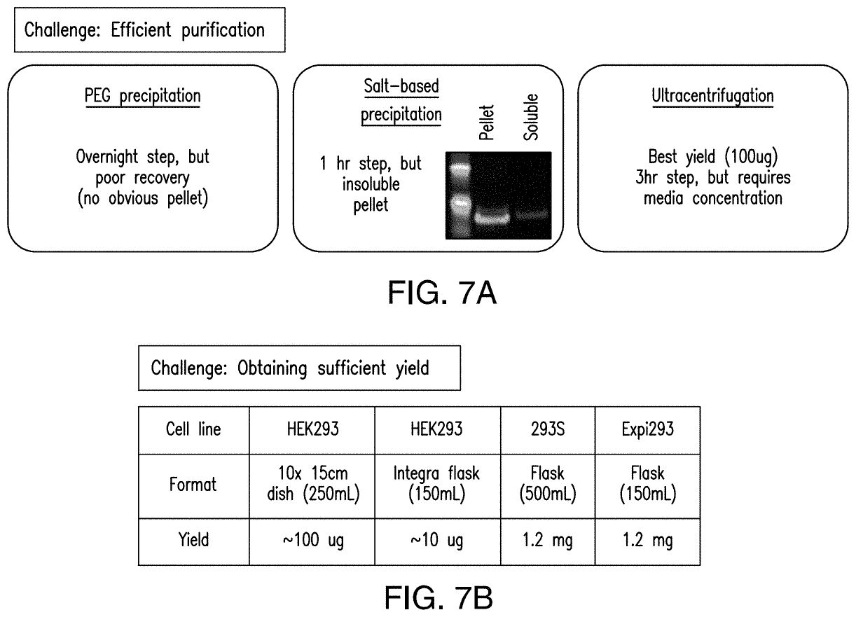

There are a number of benefits of using non-adherent cells over adherent cells for EV production. For example, use of non-adherent cells simplifies EV production as it is significantly easier to grow non-adherent cells in a single shake flask compared to the large number of tissue culture plates necessary to grow the same amount of adherent cells. Non-adherent cells are also easier and less expensive to culture, require less consumables and are easier to separate from media compared to adherent cells. In addition, the use of a non-adherent cell line such as the Expi293F™ cell line surprisingly results in high yields of vesicles even in the absence of a vesicle factor. As shown in FIG. 7B, the use of a non-adherent cell line such as the 293S cell line and the Expi293F™ cell line surprisingly resulted in higher yields of EVs compared to the adherent HEK293 cell line.

In certain embodiments, non-adherent cells are modified to express a protein of interest. For example, but not by way of limitation, a polynucleotide encoding a protein of interest is introduced into the non-adherent cell to express the protein of interest. In certain embodiments, the non-adherent cell is transfected with a polynucleotide encoding the protein of interest to express the protein of interest in the cell.

In certain embodiments, the non-adherent cells are cultured under conditions suitable for expression of the protein of interest. In certain embodiments, the non-adherent cells are cultured under conditions suitable for production of EVs. For example, but not by way of limitation, the non-adherent cells are cultured in a cell culture medium for expression of the protein of interest and/or production of EVs.

In certain embodiments, the non-adherent cells expressing a protein of interest are incubated for a suitable time to produce EVs. In certain embodiments, the non-adherent cells expressing a protein of interest are incubated for about 12 hours to about 72 hours to produce EVs. In certain embodiments, the non-adherent cells expressing a protein of interest are incubated for about 24 hours to about 64 hours to produce EVs. In certain embodiments, the non-adherent cells expressing a protein of interest are incubated for about 48 hours to produce EVs.

In certain embodiments, the EVs produced by incubation of the non-adherent cells expressing a protein of interest are subsequently purified. In certain embodiments, the EVs are purified from the cell culture medium. In certain embodiments, purification of EVs takes from about 30 minutes to about 24 hours to complete. In certain embodiments, purification of EVs takes from about 30 minutes to about 12 hours to complete. In certain embodiments, purification of EVs takes from about 30 minutes to about 5 hours to complete. In certain embodiments, purification of EVs takes from about 30 minutes to about 4 hours to complete, e.g., about 1 hour to about 4 hours to complete. In certain embodiments, purification of EVs takes about 3 hours to complete. In certain embodiments, the EVs are isolated from the medium using ultracentrifugation.

In certain embodiments, the methods described herein for producing EVs using a non-adherent cell line, e.g., in the absence of a vesicle factor, are capable of producing about 0.1 mg or more, e.g., 0.1-1.0 mg, 0.1-2.0 mg, 0.1-3.0 mg, 0.1-4.0 mg, 0.1-5.0 mg or 0.1-6.0 mg; about 0.2 mg or more; about 0.3 mg or more; about 0.4 mg or more; about 0.5 mg or more; about 0.6 mg or more; about 0.7 mg or more; about 0.8 mg or more; about 0.9 mg or more; about 1.0 mg or more, e.g., 1.0-2.0 mg, 1.0-3.0 mg, 1.0-4.0 mg, 1.0-5.0 mg or 1.0-6.0 mg; about 1.1 mg or more; about 1.2 mg or more; about 1.3 mg or more; about 1.4 mg or more; about 1.5 mg or more; about 1.6 mg or more; about 1.7 mg or more; about 1.8 mg or more; about 1.9 mg or more; about 2.0 mg or more, e.g., 2.0-3.0 mg, 2.0-4.0 mg, 2.0-5.0 mg or 2.0-6.0 mg; about 3.0 mg or more, e.g., 3.0-4.0 mg, 3.0-5.0 mg or 3.0-6.0 mg; about 4.0 mg or more, e.g., 4.0-5.0 mg or 4.0-6.0 mg; or about 5.0 mg or more of purified EVs, e.g., 5.0-6.0 mg. In certain embodiments, the methods described herein for producing EVs using a non-adherent cell line are capable of producing about 1.0 mg or more e.g., 1.0-6.0 mg, of purified EVs. In certain embodiments, the methods described herein for producing EVs using a non-adherent cell line are capable of producing the aforementioned amounts of EVs within about 24-72 hours of culturing the non-adherent cells expressing the heterologous protein. In certain embodiments, the methods described herein for producing EVs using a non-adherent cell are capable of producing the aforementioned amounts of EVs within about 24-48 hours of culturing the non-adherent cells expressing the heterologous protein. In certain embodiments, the methods described herein for producing EVs using a non-adherent cell are capable of producing the aforementioned amounts of EVs within about 48-72 hours of culturing the non-adherent cells expressing the heterologous protein.

In certain embodiments, the methods disclosed herein further comprise isolating the EVs, e.g., a plurality of EVs, from the medium. In certain embodiments, the EVs are isolated from the medium using ultracentrifugation. In certain embodiments, the EVs are isolated from the medium using a PEG precipitation. In certain embodiments, the EVs are isolated from the medium using salt-based precipitation. For example, but not by way of limitation, a method for producing EVs comprises: (a) expressing a protein of interest in a cell exposed to a vesicle factor, e.g., by expressing the vesicle factor in the cell; (b) culturing the cell in vitro in a medium to produce a plurality of EVs; and (c) isolating the EVs from the medium by ultracentrifugation, PEG precipitation and/or salt-based precipitation. In certain embodiments, a method for producing EVs comprises: (a) expressing a protein of interest in a cell exposed to a vesicle factor, e.g., by expressing the vesicle factor in the cell; (b) culturing the cell in vitro in a medium to produce a plurality of EVs; and (c) isolating the EVs from the medium by ultracentrifugation.

In certain embodiments, the cells are cultured for at least 12 hours, at least 24 hours, at least 36 hours or at least 48 hours prior to the isolation of the EVs. In certain embodiments, the cells are cultured for at least 24 hours prior to the isolation of the EVs. In certain embodiments, the cells are cultured for at least 48 hours prior to the isolation of the EVs. For example, but not by way of limitation, a method for producing EVs comprises: (a) expressing a protein of interest in a cell exposed to a vesicle factor, e.g., by expressing the vesicle factor in the cell; (b) culturing the cell in vitro in a medium for at least about 24 hours or at least about 48 hours to produce a plurality of EVs; and (c) isolating the EVs from the medium by ultracentrifugation, PEG precipitation and/or salt-based precipitation.

In certain embodiments, the protein of interest or membrane-bound antigen, e.g., membrane protein, is not fused to an exosome targeting polypeptide or peptide. In certain embodiments, the protein of interest or membrane-bound antigen, e.g., membrane protein, is not cross-linked to an exosome targeting polypeptide or peptide.

In certain embodiments, the vesicle factor is not a Gag protein. In certain embodiments, the vesicle factor is not a MLV Gag protein. In certain embodiments, the vesicle factor is not an uncleaved Gag protein. In certain embodiments, the vesicle factor is not an unmodified Gag protein. In certain embodiments, the vesicle factor is not a non-chimeric Gag protein.

In certain embodiments, the cell culture or cell suspension does not include a Gag protein. In certain embodiments using non-adherent cells, the cells are cultured in the absence of a vesicle factor, for example, in the absence of a Gag protein. In certain embodiments, non-adherent cells are used that do not comprise a polynucleotide expressing a vesicle factor. In certain embodiments, a non-adherent such as a 293S cell or an Expi293 cell is used that does not comprise a polynucleotide expressing a Gag protein, whether the Gag protein is a MLV Gag protein, an uncleaved Gag protein, a non-chimeric Gag protein or an unmodified Gag protein.

### III. EV-based ELISA Assays and Kits

The present disclosure also provides an EV-based enzyme-linked immunosorbent assay (ELISA) assay. The EV-based ELISA assay of the present disclosure has, in certain embodiments, the advantage of detecting the level of an antibody in a sample, wherein that antibody is capable of binding the native form of an antigen. In certain embodiments, the present disclosure provides methods for detecting an antibody in a sample comprising: (a) incubating the sample with a capture reagent to bind the antibody to the capture reagent, wherein the capture reagent comprises a plurality of antigen-expressing EVs, and the antibody binds specifically to the antigen; and (b) detecting the antibody bound to the capture reagent by contacting the bound antibody with a detectable antibody, wherein the detectable antibody binds specifically to the antibody. In certain embodiments, the method further comprises (c) measuring an amount of the antibody detected in (b), wherein the amount is quantitated using a standard curve. In certain embodiments, the capture reagent is immobilized to a solid phase.

In certain embodiments, the antigen is a membrane protein or a fragment thereof. In certain embodiments, the membrane protein is a single-pass membrane protein. In certain embodiments, the membrane protein is a lipid-anchored protein. In certain embodiments, the membrane protein is a multi-pass membrane protein. Any suitable methods known in the art for producing EVs can be used with the presently disclosed assays. In certain embodiments, the antigen-presenting EVs are produced in accordance with the methods disclosed in Section II. For example, but not way of limitation, an EV for use in an EV-based ELISA assay disclosed herein can comprise a vesicle factor, e.g., MLGag, Acyl.Hrs, ARRDC1 and/or ARF6, e.g., ARF6.Q67L, and an antigen.

In certain embodiments, the capture reagents disclosed herein are immobilized on a solid phase before the assay. Immobilization can be accomplished by insolubilizing the capture reagents before the assay procedure, by adsorption to a water-insoluble matrix or surface (U.S. Pat. No. 3,720,760), or non-covalent or covalent coupling (for example, using glutaraldehyde or carbodiimide cross-linking, with or without prior activation of the support with, e.g., nitric acid and a reducing agent as described in U.S. Pat. No. 3,645,852 or in Rotmans et al. J. Immunol. Methods 57:87-98 (1983)). Immobilization can be accomplished by insolubilizing the capture reagents after the assay procedure, e.g., by immunoprecipitation.

The solid phase used for immobilization can be any inert support or carrier that is essentially water insoluble and useful in immunometric assays. The inert supports can be in the forms of, e.g., surfaces, particles, porous matrices, etc. Non-limiting examples of supports include small sheets, Sephadex, polyvinyl chloride, plastic beads, and assay plates (e.g., 96-well microtiter plates) or test tubes manufactured from polyethylene, polypropylene, polystyrene, and the like, as well as particulate materials such as filter paper, agarose, cross-linked dextran, and other polysaccharides. Additionally, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 can be used with the present disclosure for capture reagent immobilization, and the contents of which are incorporated by reference in their entireties. In certain embodiments, the immobilized capture reagents are coated on a microtiter plate. In certain embodiments, the solid phase is a multi-well microtiter plate that can be used to analyze several samples at one time. In certain embodiments, the solid phase is a 96-well ELISA plate.

In certain embodiments, the capture reagents are linked onto the solid phase by a non-covalent or covalent interaction, or physical linkage as desired, to form a coated plate. Suitable techniques for attachment include those described in U.S. Pat. No. 4,376,110 and the references cited therein, and the contents of which are incorporated by reference in their entireties.

In certain embodiments, the plate or other solid phase is incubated with a cross-linking agent together with the capture reagent to link the capture reagents to the solid phase. Non-limiting examples of cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates capable of forming cross-links in the presence of light.

In certain embodiments, the coated plates are then treated with a blocking agent that binds non-specifically to and saturates the binding sites to prevent unwanted binding of the free ligand to the excess sites on the wells of the plate. Non-limiting examples of suitable blocking agents include gelatin, bovine serum albumin, egg albumin, casein, and non-fat milk.

In certain embodiments, after incubating the sample with the immobilized capture reagent, the immobilized capture reagent is contacted with a detectable antibody. In certain embodiments, the detectable antibody is a monoclonal antibody. In certain embodiments, the detectable antibody is a polyclonal antibody. In certain embodiments, the detectable antibody is directly detectable. In certain embodiments, the detectable antibody comprises a fluorimetric label or a colorimetric label. In certain embodiments, the detectable antibody is biotinylated, and the detection means is avidin or streptavidin-β-galactosidase and MUG.

The present disclosure also provides a kit for performing the presently disclosed EV-based ELISA assay. In certain embodiments, the kit for detecting the level of an antibody in a sample comprises: (a) a capture reagent comprising a plurality of antigen-presenting EVs, wherein the antigen specifically binds to the antibody; and (b) a detectable antibody that binds to the captured antibody.

In certain embodiments, the kit further comprises a solid support for the capture reagent. In certain embodiments, the solid support is provided as a separate element. In certain embodiments, the provided solid support is already coated by the capture reagent. As such, the EVs comprising membrane-bound antigen in the kit can be already immobilized on a solid support, or they can be immobilized on such support that is included with the kit or provided separately from the kit. In certain embodiments, the capture reagent is coated on a microtiter plate. In certain embodiments, the detectable antibody is a labeled antibody that can be detected directly. In certain embodiments, the detectable antibody is an unlabeled antibody that can be detected by a labeled antibody directed against the detectable antibody raised in a different species. In certain embodiments, the label is an enzyme, and the kit further comprises substrates and cofactors required by the enzyme. In certain embodiments, the label is a fluorophore, and the kit further comprises a dye precursor that provides the detectable chromophore. In certain embodiments, the detectable antibody is unlabeled, and the kit further comprise a detection means for the detectable antibody, such as a labeled antibody directed to the unlabeled antibody, preferably in a fluorimetric-detected format.

In certain embodiments, the kit further comprises instructions for carrying out the assay, and/or an antibody standard (e.g., purified antibody, preferably recombinantly produced antibody), as well as other additives such as stabilizers, washing and incubation buffers, and the like.

In certain embodiments, the components of the kit are provided in predetermined ratios, with the relative amounts of the various reagents suitably varied to obtain the desired sensitivity of the assay. In certain embodiments, the reagents are provided as dry powders (e.g., lyophilized).

### IV. Methods for Producing Antibodies Using EVs

In another aspect, the present disclosure provides methods for antibody production. Antibodies against certain antigens, e.g., membrane-bound antigens, can be challenging to make because of the difficulty to produce sufficient quantities of properly folded antigen, which can be due, in part, to cell toxicity, low expression yields, aggregation and improper folding of such antigens. See, e.g., Katzen et al. Trends Biotech. 27(8):455-460 (2009). For example, the expression of disulfide-rich (>2 disulfides) proteins can be limited because of aggregation and disulfide mispairing (see, e.g., Saez et al. Meth. Mol. Biol. 1586:155-180 (2017); Crook et al. Nat Comm. 8:2244 (2017)). In addition, the expression of membrane protein complexes (e.g., homodimer, heterodimers and homotrimers complexes) can be challenging as the transmembrane domains of one or more proteins within the complex often stabilize the higher order complex and the interactions between proteins of the complex can be weak. Further, solubilization of such membrane protein complexes in detergents can be harsh, disrupt native complex interactions or remove key interactions with the lipid environment (see, e.g., Birnbaum, et al. PNAS 111(49):17576-17581 (2014); Henrich, et al. eLife 6:e20954 (2017)). As such, various immunization approaches have been tested to produce antibodies against such challenging antigens, including immunizing mice with DNA encoding such antigens, cells expressing such antigens, denatured antigens produced by E. coli or Fc fusion antigens produced by CHO cells. The subject matter of the present disclosure is directed, in certain embodiments, to immunizing animals with EVs comprising a membrane-bound antigen, e.g., a multi-pass membrane protein, which can result in the generation of antibodies having desirable binding characteristics to challenging antigens.

In certain non-limiting embodiments, the present disclosure provides methods for immunizing an animal comprising administering a plurality of EVs comprising membrane-bound antigen to the animal to produce an antibody that binds specifically to the antigen.

In certain embodiments, the antigen is a membrane protein or a fragment thereof. In certain embodiments, the antigen is a fragment of a membrane protein. In certain embodiments, the membrane protein is a single-pass membrane protein, a lipid-anchored protein or a multi-pass membrane protein. Non-limiting examples of classes of proteins that can be used with the methods disclosed herein include receptors, e.g., G-protein coupled receptors (GPCRs), GPI-anchored proteins, ion channels, multi-transmembrane proteins, disulfide-rich extracellular domains (ECDs), unstable ECDs, multi-component complexes, e.g., homodimer protein complexes, heterodimer protein complexes, multiprotein complexes, etc. In certain embodiments, the antigen can be a protein associated with a membrane protein, e.g., a protein part of a multiprotein complex. For example, but not by way of limitation, the protein associated with a membrane protein can be a cofactor.

In certain embodiments, the membrane protein is a multi-pass membrane protein. For example, but not by way of limitation, the membrane protein spans across the membrane at least about two times, at least about three times, at least about four times, at least about five times, at least about six times, at least about seven times, at least about eight times, at least about nine times, at least about ten times, at least about eleven times or at least about twelve times. In certain embodiments, the membrane protein spans across the membrane at least seven times, e.g., a GPCR.

In certain embodiments, the membrane protein has an intracellular domain that comprises 700 amino acids or less, 650 amino acids or less, 600 amino acids or less, 550 amino acids or less, 500 amino acids or less, 450 amino acids or less, 400 amino acids or less, 350 amino acids or less, 300 amino acids or less, 250 amino acids or less, 200 amino acids or less, 150 amino acids or less, 100 amino acids or less, 95 amino acids or less, 90 amino acids or less, 85 amino acids or less, 80 amino acids or less, 75 amino acids or less, 70 amino acids or less, 65 amino acids or less, 60 amino acids or less, 55 amino acids or less, 50 amino acids or less, 45 amino acids or less, 40 amino acids or less, 35 amino acids or less, 30 amino acids or less, 25 amino acids or less, 20 amino acids or less, 15 amino acids or less, 10 amino acids or less or 5 amino acids or less. For example, but not by way of limitation, the membrane protein has an intracellular domain that comprises 400 amino acids or less. In certain embodiments, the membrane protein has an intracellular domain that comprises 700 amino acids or less, 600 amino acids or less, 500 amino acids or less, 400 amino acids or less, 300 amino acids or less, 200 amino acids or less or 100 amino acids or less if a Gag, e.g., MLGag, vesicle factor is used to generate EVs displaying the membrane protein of interest, e.g., membrane protein of interest. In certain embodiments, the membrane protein has an intracellular domain that comprises 200 amino acids or less if a Gag, e.g., MLGag, vesicle factor is used to generate EVs displaying the membrane protein of interest, e.g., membrane protein of interest.

In certain embodiments, the membrane protein is an ion channel. In certain embodiments, the ion channel is a cation channel. For example, but not by way of limitation, the membrane protein is a potassium ion channel, a sodium ion channel or a calcium ion channel. In certain embodiments, the ion channel is a sodium ion channel.

Methods known in the art for making EVs can be used with the methods disclosed herein. For example, but not by way of limitation, methods known in the art for making EVs displaying a protein of interest, which comprises the antigen, can be used with the antibody generation methods disclosed herein. In certain embodiments, the EVs are produced using the methods disclosed in Section II of the present disclosure. In certain embodiments, the antigen is located on the membrane of the EVs, where the conformation of the antigen is substantially similar to its conformation, e.g., native conformation, on a cell membrane.

In certain non-limiting embodiments, the present disclosure provides methods for producing an antibody against a protein of interest. In certain embodiments, the method includes immunizing an animal by administering a plurality of EVs comprising an antigen, e.g., a protein of interest, e.g., a membrane protein of interest, to the animal to produce an antibody that binds specifically to the antigen.

In certain embodiments, immunizing an animal comprises injecting the EVs into the animal. Immunization can involve one or more administrations of the EVs to an animal. In certain embodiments, the methods comprise administration of the EVs to the animal 1, 2, 3, 4, 5, 6, 7, 8, 9 or 10 or more times. In certain embodiments, the EVs are administered to the animal from about 3 to about 6 times. In certain embodiments, the EVs are administered to the animal on week 0, week 2 and week 4.

In certain embodiments, immunization of the animal can be monitored by FACS to detect the level of target-specific antibodies being generated. If a suitable titer is detected (e.g., by detection of a FACS response at a 1:1000 serum dilution), generation of a monoclonal antibody can be initiated as described herein, e.g., by B cell cloning or hybridoma generation.

In certain embodiments, the methods further comprise collecting an antiserum from the animal after the EV administration, where the antiserum comprises the antibody produced by the animal.

In certain embodiments, the EVs are administered to the animal along with an adjuvant. Non-limiting examples of adjuvants include Freund's adjuvants (optionally comprising mycobacterium or its components to form Freund's complete adjuvant (FCA)), Ribi adjuvants, Titermax adjuvants, specol adjuvants, and aluminum salt adjuvants. In certain embodiments, the adjuvant is a Ribi adjuvant. Ribi adjuvants are oil-in-water emulsions, wherein the antigen (e.g., antigen-presenting EVs) is mixed with metabolizable oil (squalene), which is emulsified in a saline solution containing Tween 80. In certain embodiments, Ribi adjuvant also contains refined mycobacterial product that acts as immunostimulants and a gram-negative bacterial product monophosphoryl lipid A. In certain embodiments, Ribi interacts with membranes of immune cells resulting in cytokine induction that enhances antigen uptake, processing and presentation. In certain embodiments, a method for producing an antibody against a protein of interest includes administering to an animal a plurality of EVs displaying the protein of interest in combination with an adjuvant.

In certain embodiments, the methods further comprise administering a boost to the animal. For example, but not by way of limitation, a method for producing an antibody against a protein of interest includes administering to an animal a plurality of EVs displaying the protein of interest in combination with a boost. The boost can enhance the immune response in the animal and thus increase the quantity and quality of the antibody produced by the animal. In certain embodiments, the boost comprises a polynucleotide which encodes the antigen or a fragment of the antigen. In certain embodiments, the polynucleotide is a DNA. In certain embodiments, the boost comprises a polypeptide or a protein that comprises the antigen or a fragment thereof. In certain embodiments, the boost is administered simultaneously with the EVs. In certain embodiments, the boost is administered about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 day, about 17 days, about 18 days, about 19 days, about 20 days, about 21 days, about 22 days, about 23 days about 24 days, about 25 days, about 26 days, about 27 days, about 28 days, about 29 days, and/or about 30 days after the EVs are administered to the animals. In certain embodiments, the boost is administered about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 day, about 17 days, about 18 days, about 19 days, about 20 days, about 21 days, about 22 days, about 23 days about 24 days, about 25 days, about 26 days, about 27 days, about 28 days, about 29 days, and/or about 30 days after the first dose of EVs is administered to the animals. For example, but not by way of limitation, the boost is administered about 14 days after the EVs, e.g., the first dose of EVs, are administered to the animals. In certain embodiments, the boost is administered about 21 days after the EVs, e.g., the first dose of EVs, are administered to the animals. In certain embodiments, the EVs can be administered to an animal in combination with an adjuvant and a boost.

Any animals known in the art for immunization and antibody production can be used with the methods disclosed herein. Non-limiting examples of animals which can be used with the methods disclosed herein include non-human primates such as Old-World monkey (e.g., baboon or macaque, including Rhesus monkey and cynomolgus monkey, see U.S. Pat. No. 5,658,570), birds (e.g., chickens); rabbits, goats, sheep, cows, horses, pigs, donkeys, llamas, alpacas, and dogs. In certain embodiments, the animal is a rodent. A “rodent” is an animal belonging to the Rodentia order of placental mammals. Non-limiting examples of rodents which can be used herein include mice, rats, guinea pigs, squirrels, hamsters, and ferrets. In certain embodiments, the animal for immunization is a mouse.

The EVs comprising membrane-bound antigens can be delivered to various cells of the animal body, including for example, muscle, skin, brain, lung, liver, spleen, or to the cells of the blood. Administration of EVs comprising membrane-bound antigens is not limited to a particular route or site. Non-limiting examples of the administering routes include intramuscular, intradermal, epidermal, intra pinna, oral, vaginal, and nasal. In certain embodiments, the EVs comprising membrane-bound antigens are administered to the animals intramuscularly, intradermally or epidermally. In certain embodiments, the EVs are delivered to the tissues of muscle, skin or mucous membranes.

In certain embodiments, the methods disclosed herein further comprise obtaining immune cells from the immunized animal disclosed above, where the immune cells produce or are capable of producing polyclonal antibodies. Such immune cells can then be fused with myeloma cells using a suitable fusing agent, such as polyethylene glycol or Sendai virus, to form a hybridoma cell (Godmg, Monoclonal Antibodies: Principles and Practice, pp. 59-103, Academic Press, 1986). Alternatively or additionally, the methods disclosed herein can comprise generating the antibodies by B cell culture cloning or the production of immune phage libraries. See, e.g., Bazan et al. Hum. Vaccin. Immunother. 8(12):1817-1828 (2012); Carbonetti et al. J. Immunol. Methods 448:66-73 (2017), the contents of which are incorporated by reference herein.

The hybridoma cells thus prepared can be seeded and grown in a suitable culture medium that preferably contains one or more substances that inhibits the growth or survival of the unfused, parental myeloma cells. For example, if the parental myeloma cells lack the enzyme hypoxanthme guanme phosphoribosyl transferase (HGPRT or HPRT), the culture medium for the hybridomas typically includes hypoxanthme, ammopterm, and thymide (HAT medium), which prevents the growth of HGPRT-deficient cells. Non-limiting examples of myeloma cell lines are murine myeloma lines, such as those derived from MOPC-21 and MPC-11 mouse tumors, and P3X63AgU.1, SP-2 or X63-Ag8-653 cells; rat myeloma cell line 210-RCY3.Ag1.2.3; and human myeloma and mouse-human heteromyeloma cell lines.

Alternatively, hybridoma cell lines can be prepared from the immune cells of the immunized animal in other ways, e.g., by immortalizing the immune cells with a virus (e.g., with Epstein Barr Virus) or with an oncogene in order to produce an immortalized cell line producing the monoclonal antibody of interest. See, also, Babcock et al. PNAS (USA), 93:7843-7848 (1996), concerning production of monoclonal antibodies by cloning immunoglobulin cDNAs from single cells producing specific antibodies for yet another strategy for preparing monoclonal antibodies using immune cells of the immunized animal.

In certain embodiments, the methods disclosed herein further comprises a screening step to identify one or more monoclonal antibodies capable of binding to each antigen. In certain embodiments, the methods further comprise screening for antibodies which bind to the antigen with which the animal has been immunized. In certain embodiments, the screening can be carried out using culture supernatant and/or purified antibodies from cloned hybridoma cells. The binding specificity of monoclonal antibodies produced by hybridoma cells can, for example, be determined in an immuno-assay. Non-limiting examples of immuno-assays include ELISAs, radioimmunoassays (RIAs), and FACS assays. In certain embodiments, the EV-based ELISA disclosed herein can be used for antibody screening.

In certain embodiments, the methods disclosed herein allow for the sorting antibody-producing cells. For example, antibody-producing cells can, in certain embodiments, be incubated with a plurality of EVs wherein the plurality of EVs comprise: (1) a first population of EVs comprising a membrane-bound antigen and a first detectable marker, wherein a subset of the antibody-producing cells bind specifically to the membrane-bound antigen; and (2) a second population of EVs lacking the membrane-bound antigen of the first population of EVs, but which comprise a second detectable marker distinguishable from the first marker. In certain embodiments, the antibody-producing cells can then be sorted based on their binding to either the first population of EVs comprising the first marker, or to a combination of the of the first population of EVs comprising first marker and the second population of EVs comprising the second marker. In certain embodiments, the first and second detectable markers are fluorescent markers. In certain embodiments, the first and second fluorescent markers are fluorescent proteins. In certain embodiments, the sorting is performed by FACS. In certain embodiments, the antibody-producing cells are B cells. In certain embodiments, the antibody-producing cells are hybridoma cells.

### V. Methods and Compositions for Diagnostics and Detection

In certain embodiments, an antibody disclosed herein, e.g., an antibody generated by a method disclosed herein, can be useful for detecting the presence of an antigen in a biological sample. The term “detecting” as used herein encompasses quantitative or qualitative detection.

In certain embodiments, an antibody for use in a method of diagnosis or detection is provided, e.g., an antibody generated using a method disclosed herein. In a further aspect, a method of detecting the presence of antigen in a biological sample is provided. In certain embodiments, the method comprises contacting the biological sample with an antibody as described herein under conditions permissive for binding of the antibody to its corresponding antigen, and detecting whether a complex is formed between the antibody and the associated antigen. Such method can be an in vitro or in vivo method. In certain embodiments, an antibody is used to select subjects eligible for therapy with an antibody, e.g., where antigen is a biomarker for selection of patients.

In certain embodiments, labeled antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### VI. Pharmaceutical Compositions

In a further aspect, the present disclosure provides pharmaceutical compositions comprising any of the antibodies disclosed herein, e.g., for use in any of the below therapeutic methods. For example, but not by way of limitation, the antibody is generated using the methods disclosed herein. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Pharmaceutical compositions of an antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO2006/044908, the latter compositions including a histidine-acetate buffer.

The pharmaceutical composition herein can also contain more than one active ingredient as necessary for the particular indication being treated, preferably those with complementary activities that do not adversely affect each other. Such active ingredients are suitably present in combination in amounts that are effective for the purpose intended.

Active ingredients can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Pharmaceutical compositions for sustained-release can be prepared. Suitable examples of sustained-release preparations include semipermeable matrices of solid hydrophobic polymers containing the antibody, which matrices are in the form of shaped articles, e.g. films, or microcapsules.

The pharmaceutical compositions to be used for in vivo administration are generally sterile. Sterility can be readily accomplished, e.g., by filtration through sterile filtration membranes.

### VII. Therapeutic Methods and Routes of Administration

Any of the antibodies provided herein can be used in therapeutic methods. For example, but not by way of limitation, the present disclosure provides an antibody generated by a method of the present disclosure for use in a therapeutic method.

In one aspect, an antibody disclosed herein, e.g., an antibody generated by a method of the present disclosure, for use as a medicament is provided. In further aspects, an antibody disclosed herein, e.g., an antibody generated by a method of the present disclosure, for use in treating a disease is provided. In certain embodiments, an antibody disclosed herein for use in a method of treatment is provided.

In certain embodiments, the present disclosure provides an antibody disclosed herein, e.g., an antibody generated by a method of the present disclosure, is for use in a method of treating an individual having a disease. In certain embodiments, the method includes administering to the individual an effective amount of the antibody disclosed herein. In certain embodiments, the method further comprises administering to the individual an effective amount of at least one additional therapeutic agent (e.g., one, two, three, four, five, or six additional therapeutic agents).

In a further aspect, the present disclosure provides for the use of an antibody disclosed herein, e.g., an antibody generated by a method of the present disclosure, in the manufacture or preparation of a medicament. In certain embodiments, the medicament is for treatment of a disease. In certain embodiments, the medicament is for use in a method of treating a disease comprising administering to an individual having the disease an effective amount of the medicament. In certain embodiments, the method further comprises administering to the individual an effective amount of at least one additional therapeutic agent.

In a further aspect, the present disclosure provides a method for treating a disease. In certain embodiments, the method comprises administering to an individual having such disease an effective amount of an antibody disclosed herein. In certain embodiments, the method further comprises administering to the individual an effective amount of at least one additional therapeutic agent.

An “individual” according to any of the above embodiments can be a human.

In a further aspect, the present disclosure provides pharmaceutical compositions comprising any of the antibodies provided herein, e.g., for use in any of the above therapeutic methods. In certain embodiments, a pharmaceutical composition comprises any of the antibodies provided herein and a pharmaceutically acceptable carrier. In certain embodiments, a pharmaceutical composition comprises any of the antibodies provided herein and at least one additional therapeutic agent, e.g., as described below.

Antibodies of the present disclosure can be used either alone or in combination with other agents in a therapy. For instance, an antibody of the present disclosure can be co-administered with at least one additional therapeutic agent

Such combination therapies noted above encompass combined administration (where two or more therapeutic agents are included in the same or separate pharmaceutical compositions), and separate administration, in which case, administration of the antibody of the present disclosure can occur prior to, simultaneously, and/or following, administration of the additional therapeutic agent or agents. In certain embodiments, administration of an antibody described herein and administration of an additional therapeutic agent occur within about one month, or within about one, two or three weeks, or within about one, two, three, four, five, or six days, of each other. In certain embodiments, the antibody described herein and additional therapeutic agent are administered to the patient on Day 1 of the treatment. Antibodies of the present disclosure can also be used in combination with radiation therapy.

An antibody of the present disclosure (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the present disclosure would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the present disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) can be administered to the patient. Such doses can be administered intermittently, e.g. every week or every three weeks (e.g. such that the patient receives from about two to about twenty, or e.g. about six doses of the antibody). An initial higher loading dose, followed by one or more lower doses can be administered. An exemplary dosing regimen comprises administering. However, other dosage regimens can be useful. The progress of this therapy is easily monitored by conventional techniques and assays.

### VIII. Articles of Manufacture

In another aspect of the present disclosure, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers can be formed from a variety of materials such as glass or plastic. The container holds a composition which is by itself or combined with another composition effective for treating, preventing and/or diagnosing the condition and can have a sterile access port (for example the container can be an intravenous solution bag or a vial having a stopper pierceable by a hypodermic injection needle).

At least one active agent in the composition is an antibody of the present disclosure, e.g., an antibody generated by the methods of the present disclosure. The label or package insert indicates that the composition is used for treating the condition of choice. Moreover, the article of manufacture can comprise (a) a first container with a composition contained therein, wherein the composition comprises an antibody of the present disclosure, e.g., an antibody generated by the methods of the present disclosure; and (b) a second container with a composition contained therein, wherein the composition comprises a further cytotoxic or otherwise therapeutic agent. The article of manufacture in this embodiment of the present disclosure can further comprise a package insert indicating that the compositions can be used to treat a particular condition. Alternatively, or additionally, the article of manufacture can further comprise a second (or third) container comprising a pharmaceutically-acceptable buffer, such as bacteriostatic water for injection (BWFI), phosphate-buffered saline, Ringer's solution and dextrose solution. It can further include other materials desirable from a commercial and user standpoint, including other buffers, diluents, filters, needles, and syringes.

### IX. Exemplary Embodiments

A. In certain non-limiting embodiments, the presently disclosed subject matter provides for a method for producing an antibody that specifically binds to a protein, wherein the method comprises:

(a) producing a plurality of extracellular vesicles (EVs) comprising a heterologous protein by (i) expressing the heterologous protein in a cell exposed to a vesicle factor, (ii) culturing the cell in a medium and (iii) isolating the plurality of EVs comprising the heterologous protein from the medium, wherein the vesicle factor is selected from the group consisting of Acyl.Hrs, ARRDC1, ARF6 and a combination thereof;

(b) immunizing an animal by administering the plurality of EVs to the animal; and

(c) isolating an antibody that binds to the protein from the animal.

A1. The foregoing method of A, wherein the cell is a non-adherent cell.

A2. The foregoing method of A and A1, wherein expressing the vesicle factor and the protein in a cell comprises introducing one or more polynucleotides encoding the vesicle factor and the protein in the cell.

A3. The foregoing method of A2, wherein the vesicle factor and the protein are encoded by a single polynucleotide.

A4. The foregoing method of A2, wherein the vesicle factor is encoded by a first polynucleotide and the protein is encoded by a second polynucleotide.

B. In certain non-limiting embodiments, the presently disclosed subject matter provides for a method for producing an antibody that specifically binds to a protein, wherein the method comprises: (a) producing a plurality of extracellular vesicles (EVs) comprising a heterologous protein by (i) expressing the heterologous protein in a cell, (ii) culturing the cell in a medium and (iii) isolating the plurality of EVs comprising the heterologous protein from the medium, wherein the cell is a non-adherent cell; (b) immunizing an animal by administering the plurality of EVs to the animal; and (c) isolating an antibody that binds to the heterologous protein from the animal.

B1. The foregoing method of B, wherein producing the plurality of EVs further comprises expressing a heterologous vesicle factor in the cell.

B2. The foregoing method of B1, wherein the vesicle factor is selected from the group consisting of MLGag, Acyl.Hrs, ARRDC1, ARF6 and a combination thereof.

B3. The foregoing method of A-B2, wherein the heterologous protein is a membrane protein.

B4. The foregoing method of B3, wherein the membrane protein is a single-pass membrane protein.

B5. The foregoing method of B3, wherein the membrane protein is a multi-pass membrane protein.

B6. The foregoing method of B3, wherein the membrane protein is a member of a protein complex.

B7. The foregoing method of B3, wherein the membrane protein is not a transmembrane protein and is a member of a protein complex.

B8. The foregoing method of A1-B7, wherein the non-adherent cell is a 293S cell or an Expi293™ cell.

B9. The foregoing method of A-B8, wherein the EVs are isolated from the medium by ultracentrifugation.

B10. The foregoing method of A-B9, wherein the plurality of EVs is administered to the animal on week 0, week 2 and week 4.

B11. The foregoing method of A-B10, further comprising administering an adjuvant to the animal concurrently with the EVs.

B12. The foregoing method of B11, wherein the adjuvant is a Ribi adjuvant.

B13. The foregoing method of A-B12, further comprising administering a boost to the animal to enhance an immune response in the animal to the protein.

B14. The foregoing method of B13, wherein the boost comprises the protein, a polynucleotide encoding the protein or a combination thereof.

B15. The foregoing method of B14, wherein the boost comprises the protein.

B16. The foregoing method of B14, wherein the boost comprises a polynucleotide encoding the protein.

B17. The foregoing method of A-B16, wherein the antibody is a monoclonal antibody.

B18. The foregoing method of A-B17, wherein the antibody is a human, humanized or chimeric antibody.

C. In certain non-limiting embodiments, the presently disclosed subject matter provides an isolated antibody or an antigen-binding portion thereof produced by the method of any one of A-B18.

D. In certain non-limiting embodiments, the presently disclosed subject matter provides an isolated nucleic acid encoding the antibody or antigen-binding portion thereof of C.

E. In certain non-limiting embodiments, the presently disclosed subject matter provides a host cell comprising the nucleic acid of D.

F. In certain non-limiting embodiments, the presently disclosed subject matter provides for a method of producing an antibody or antigen-binding portion thereof, wherein the method comprises culturing the host cell of E under conditions suitable for expression of the antibody.

F1. The foregoing method of F, further comprising recovering the antibody from the host cell.

G. In certain non-limiting embodiments, the presently disclosed subject matter provides a pharmaceutical composition comprising the isolated antibody or antigen-binding portion thereof of C and a pharmaceutically acceptable carrier.

G1. The foregoing pharmaceutical composition of G, further comprising an additional therapeutic agent.

H. The foregoing isolated antibody or antigen-binding portion thereof of C for use as a medicament.

I. The foregoing isolated antibody or antigen-binding portion thereof of C for use in treating a disease.

J. In certain non-limiting embodiments, the presently disclosed subject matter provides for a use of the isolated antibody or antigen-binding portion thereof of C in the manufacture of a medicament.

K. In certain non-limiting embodiments, the presently disclosed subject matter provides for a method of treating an individual having a disease, wherein the method comprises administering to the individual an effective amount of the isolated antibody or antigen-binding portion thereof of C.

K1. The foregoing method of K, further comprising administering an additional therapeutic agent to the individual.

L. In certain non-limiting embodiments, the presently disclosed subject matter provides for a method of treating an individual having a disease comprising administering to the individual the pharmaceutical composition of G or G1.

M. In certain non-limiting embodiments, the presently disclosed subject matter provides for a method for detecting an antibody in a sample, wherein the method comprises:

(a) incubating a sample with a capture reagent, wherein the capture reagent comprises a plurality of EVs comprising a membrane-bound antigen, and the antibody binds specifically to the membrane-bound antigen; and

(b) contacting the antibody binding to the capture reagent with a detectable antibody to detect the bound antibody, wherein the detectable antibody binds specifically to the antibody,

wherein the plurality of EVs are generated by (i) expression of the membrane-bound antigen in a cell, (ii) culturing the cell in vitro in a medium to produce the plurality of EVs displaying the membrane-bound antigen and (iii) isolating the plurality of EVs displaying the membrane-bound antigen from the medium, and

wherein the cell is exposed to a vesicle factor selected from the group consisting of Acyl.Hrs, ARRDC1, ARF6 and a combination thereof and/or the cell is a non-adherent cell.

M1. The foregoing method of M, further comprising (c) measuring the amount of the antibody detected in (b), wherein the amount is quantitated using a standard curve.

M2. The foregoing method of M or M1, wherein the sample is a plasma, a serum or a urine sample.

M3. The foregoing method of M-M2, wherein the capture reagent immobilized on a solid support.

M4. The foregoing method of M3, wherein the solid support is a microtiter plate.

M5. The foregoing method of M-M4, wherein the detectable antibody is fluorescently labeled.

M6. The foregoing method of M-M5, wherein the membrane-bound antigen is a membrane protein or a fragment thereof.

N. In certain non-limiting embodiments, the presently disclosed subject matter provides for a method for sorting antibody-producing cells, wherein the method comprises:

(a) incubating the antibody-producing cells with a plurality of EVs wherein the plurality of EVs comprise: i. a first population of EVs comprising a membrane-bound antigen and a first detectable marker, wherein a subset of the antibody-producing cells bind specifically to the membrane-bound antigen; and ii. a second population of EVs lacking the membrane-bound antigen but comprising a second detectable marker distinguishable from the first marker; and

(b) sorting the antibody-producing cells based on their binding to either the first population of EVs or to a combination of the first population of EVs and the second population of EVs,

wherein the first population of EVs is generated by (i) expression of the membrane-bound antigen and the first detectable marker in a first cell, (ii) culturing the first cell in vitro in a medium to produce the plurality of EVs displaying the membrane-bound antigen and (iii) isolating the plurality of EVs displaying the membrane-bound antigen from the medium,

wherein the second population of EVs is generated by (i) expression of the second detectable marker in a second cell, (ii) culturing the second cell in vitro in a medium to produce the plurality of EVs comprising the second detectable marker and (iii) isolating the plurality of EVs displaying the second detectable marker from the medium, and

wherein (i) the first cell and/or the second cell is contacted with a vesicle factor selected from the group consisting of Acyl.Hrs, ARRDC1, ARF6 and a combination thereof and/or (ii) the first and/or second cell is a non-adherent cell.

N1. The foregoing method of N, wherein the first and second detectable markers are fluorescent markers.

N2. The foregoing method of N1, wherein the first and second fluorescent markers are fluorescent proteins.

N3. The foregoing method of N-N2, wherein the sorting is performed by fluorescence-activated cell sorting.

N4. The foregoing method of N-N3, wherein the antibody-producing cells are B cells.

N5. The foregoing method of N-N4, wherein the antibody-producing cells are hybridoma cells.

O. In certain non-limiting embodiments, the presently disclosed subject matter provides for a method for producing a plurality of extracellular vesicles (EVs) displaying a heterologous protein, wherein the method comprises:

(a) expressing the heterologous protein in a cell;

(b) culturing the cell in a medium; and

(c) isolating the plurality of EVs comprising the heterologous protein from the medium,

wherein the cell is exposed to a vesicle factor selected from the group consisting of Acyl.Hrs, ARRDC1, ARF6 and a combination thereof, and/or the cell is a non-adherent cell.

O1. The foregoing method of O, wherein the heterologous protein is a membrane protein.

O2. The foregoing method of O1, wherein the membrane protein is a single-pass membrane protein.

O3. The foregoing method of O1, wherein the membrane protein is a multi-pass membrane protein.

O4. The foregoing method of O1-O3, wherein the membrane protein is a member of a protein complex.

O5. The foregoing method of M-O4, wherein the non-adherent cell is a 293S cell or an Expi293™ cell.

O6. The foregoing method of M-O5, wherein the plurality of EVs are isolated from the medium by ultracentrifugation.

P. In certain non-limiting embodiments, the presently disclosed subject matter provides for a kit for detecting an antibody in a sample, wherein the kit comprises:

(a) a capture reagent that comprises a plurality of EVs comprising membrane-bound antigen, wherein the antibody to be detected binds specifically to the antigen; and

(b) a detectable antibody binds specifically to the antibody to be detected,

wherein the plurality of EVs are generated by (i) expression of the membrane-bound antigen in a cell, (ii) culturing the cell in vitro in a medium to produce the plurality of EVs displaying the membrane-bound antigen and (iii) isolating the plurality of EVs displaying the membrane-bound antigen from the medium, and

wherein the cell is exposed to a vesicle factor selected from the group consisting of Acyl.Hrs, ARRDC1, ARF6 and a combination thereof and/or the cell is a non-adherent cell.

P1. The foregoing kit of P, wherein the plurality of EVs are immobilized on a solid support.

P2. The foregoing kit of P or P1, wherein the solid support is a microtiter plate.

P3. The foregoing kit of P-P2, wherein the detectable antibody is fluorescently labeled.

P4. The foregoing kit of P-P3, wherein the membrane-bound antigen is a membrane protein or a fragment thereof.

### EXAMPLES

The presently disclosed subject matter will be better understood by reference to the following Examples, which are provided as exemplary of the presently disclosed subject matter, and not by way of limitation.

### Example 1: Identification of Vesicle Factors that Produce Vesicles with MP-X

293T cells were co-transfected with a target protein human G-protein coupled receptor (Membrane Protein (MP)-X) and a vesicle factor that was selected from hARRDC1, Acyl-Hrs, ARF6.Q67L, RhoA.F30L, constitutively active ROCK, MemPro, and MLGag. EVs were generated as shown in FIG. 3. Cell lysates were collected for testing expression of the target protein and vesicle factor. Culture medium was collected and processed. EVs were collected from the culture medium by ultracentrifugation. Purified EVs were analyzed by Western blot using anti-FLAG antibody (1:1000 dilution M2 Sigma F3165).

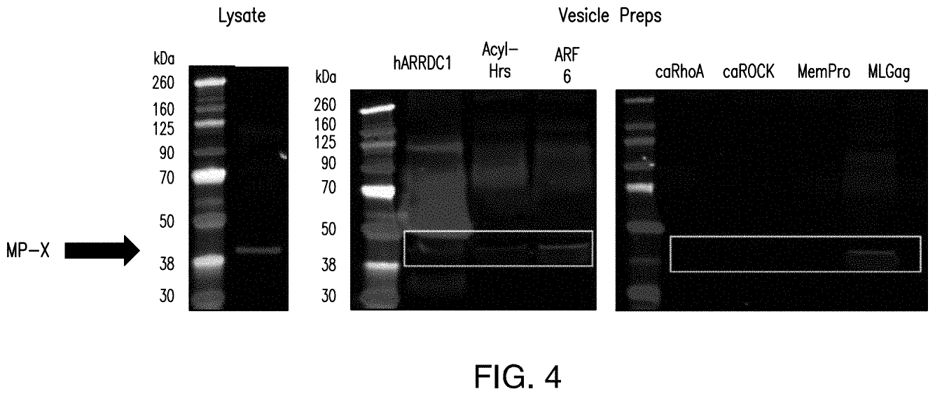

As shown in FIG. 4, MP-X was expressed in EVs produced from cells transfected with vesicle factors hARRDC1, Acyl.Hrs, ARF6.Q67L, or MLGag, but not in EVs produced from cells transfected with vesicle factors RhoA.F30L, constitutively active

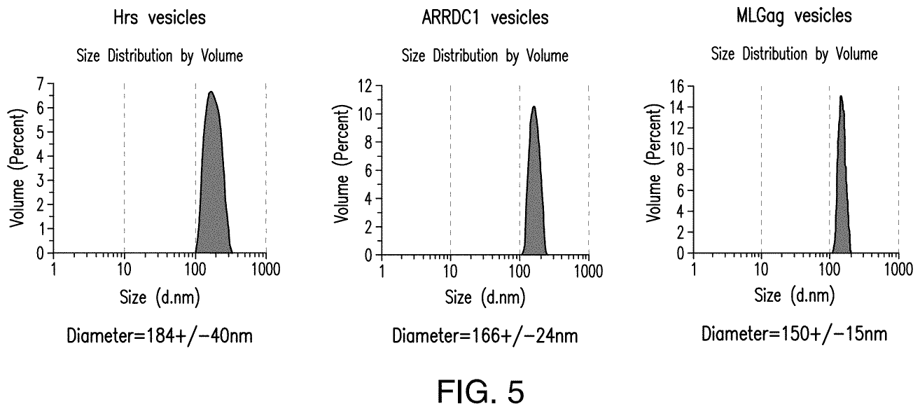

ROCK and MemPro. In addition, Dynamic Light Scattering (DLS) showed that EVs produced from cells transfected with hARRDC1, Acyl.Hrs, or MLGag had uniform vesicle sizes (FIG. 5).

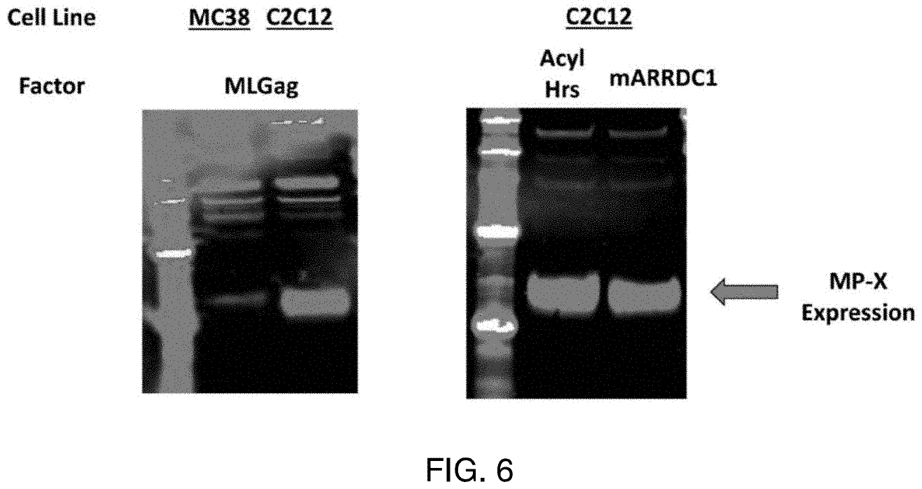

Next, the effectiveness of these vesicle factors in inducing EVs was tested with murine cells. Murine colon cancer cells MC38 and murine myoblasts C2C12 were co-transfected with MP-X and a vesicle factor selected from MLGag, Acyl.Hrs, and mARRDC1, to produce EVs. After purifying EVs from the culture medium by ultracentrifuge, the EVs were analyzed by Western Blot (anti-FLAG primary antibody, 1:1000 dilution M2 Sigma F3165) to detect the presence of MP-X. FIG. 6 showed that MP-X levels were high in EVs produced from cells transfected with vesicle factor MLGag, Acyl.Hrs, or mARRDC1. MP-X was not detected in EVs produced from cells not transfected with vesicle factors.

### Example 2: Improved Methods to Enable Rapid EV Generation with High Yields

One challenge in EV production is to purify EVs from the culturing medium efficiently (FIG. 7A). The PEG precipitation method had very poor recovery, producing no obvious pellet, and requiring overnight step. The salt-based precipitation only required an hour but produced insoluble pellet. The present study found that ultracentrifugation purification had the most efficient purification of all three methods, and only required 3 hours.

Another challenge in EV production is to select cell lines that have sufficient yields. After comparing the yields of a number of cell lines, the present study found that Expi293™ and 293S cells produced the highest yields of all four cell lines (FIG. 7B). JetPEI was used as the transfection method.

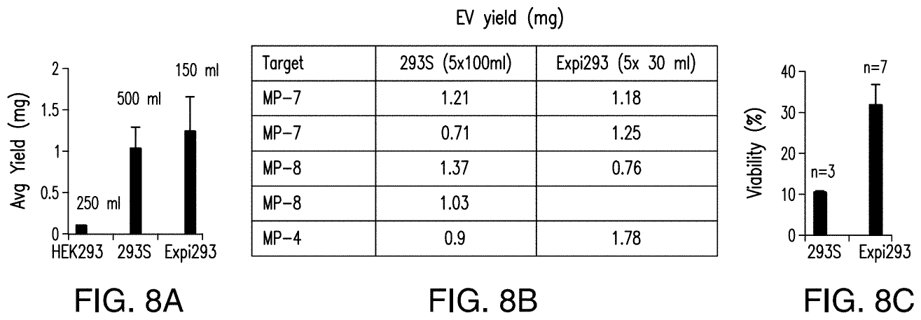

The present study also compared the EV yields and target protein (i.e., protein of interest) expression between the Expi293F™ cell line and 293S cell line. FIGS. 8A-8B showed that the Expi293 cell line had higher average yield than the 293S cell line, when using MLGag as the vesicle factor. FIG. 8C showed that viability of cells is better in the Expi293F™ cell line than in the 293S cell line.

The doubling time for the Expi293F™ cell line and 293S cell line were very similar at about 24 hours. Post transfection cell growth was reduced in both cell lines. 293S cell line can have one doubling during the production phase, whereas Expi293F™ cell lines had several doublings during the production phase.

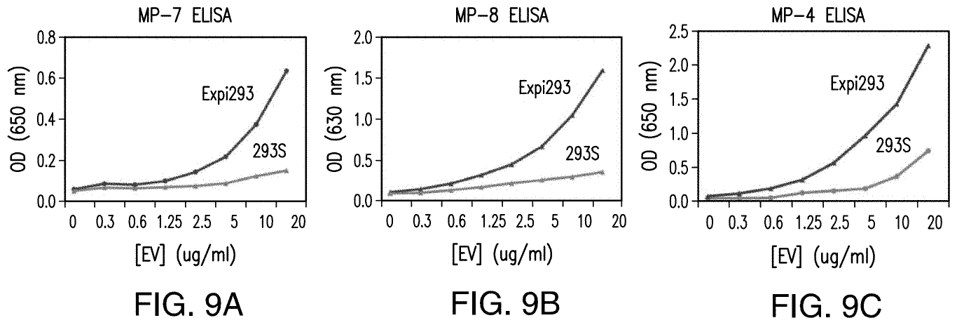

Additionally, the presence of each target protein in EVs was measured using ELISA. FIGS. 9A-9C showed that the EVs from the Expi293F™ cell line had higher target protein concentration than EVs from the 293S cell line.

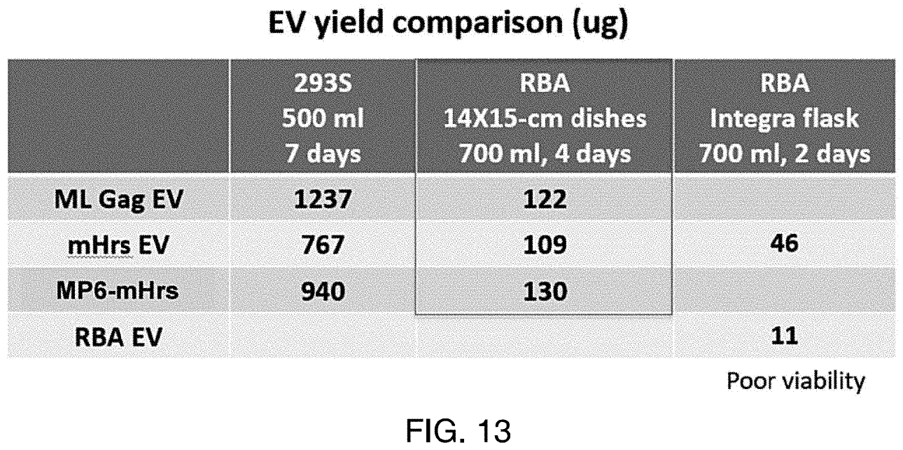

The present study also tested whether rat RBA cells can be used for EV production. RBA cell line is a mammary gland adenocarcinoma cell line originated from SD rat. It was found that RBA cells can produce EVs, but the yields were very low when compared with 293S cell line (FIG. 13).

### Example 3: EVs Enabled ELISA-Based Detection of FACS+ Antibodies Against Complex Membrane Proteins

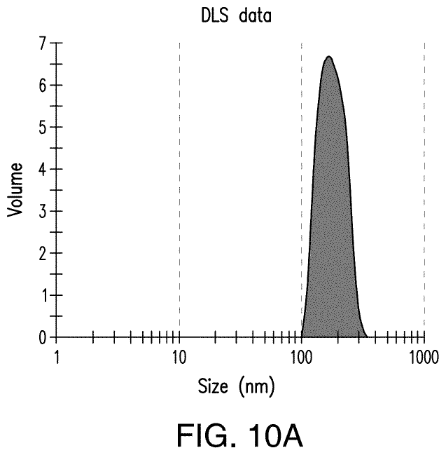

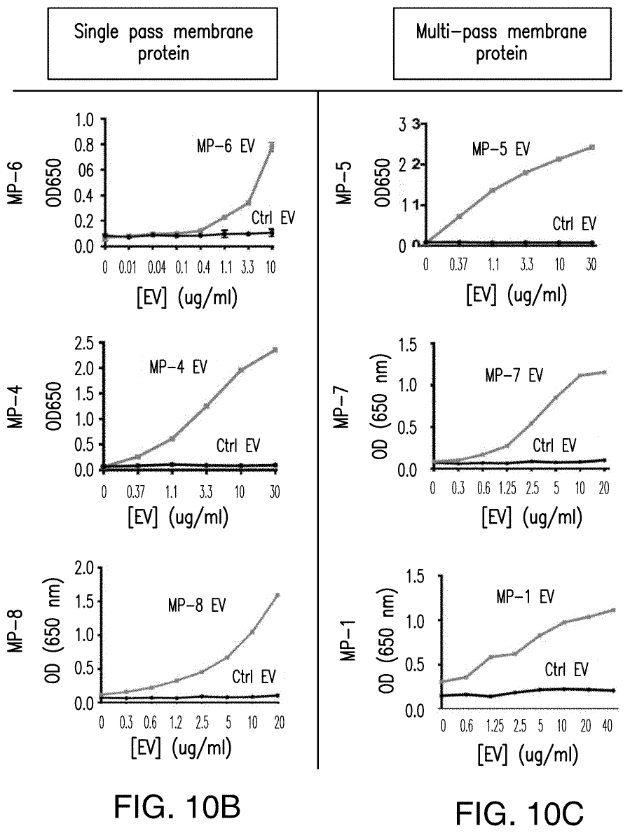

The present study found that vesicle factors MLGag, Acyl.Hrs, ARRDC1, and ARF6 helped generate well-defined particles (diameter of 184±40 nm) using Expi293F™ cell line (FIG. 10A). Additionally, EVs enabled ELISA-based detection of single-pass membrane proteins and multi-pass membrane proteins using FACS+ antibodies against those proteins that were incorporated by EVs (FIG. 10B-10C).

### Example 4: Identification of Cell Lines for Screening Antibodies from Expi293F™ EV Immunized Rats, Rabbits, Llamas, and Mice

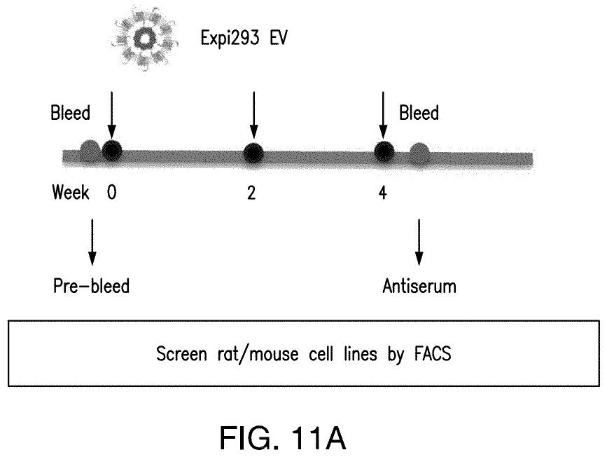

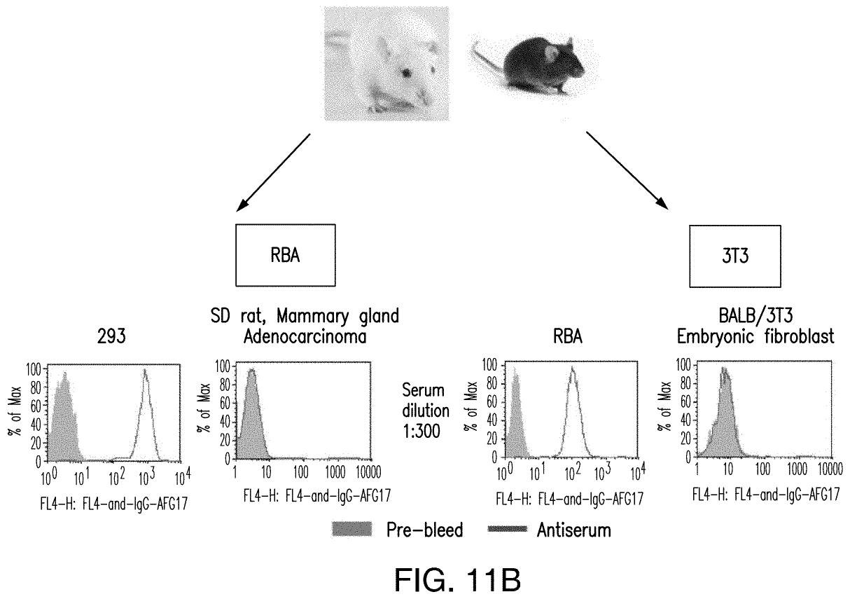

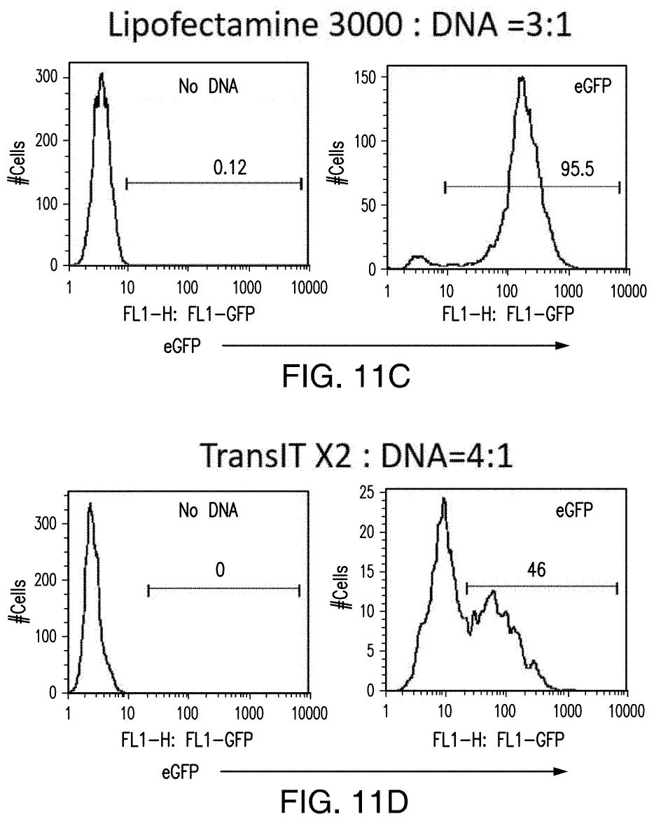

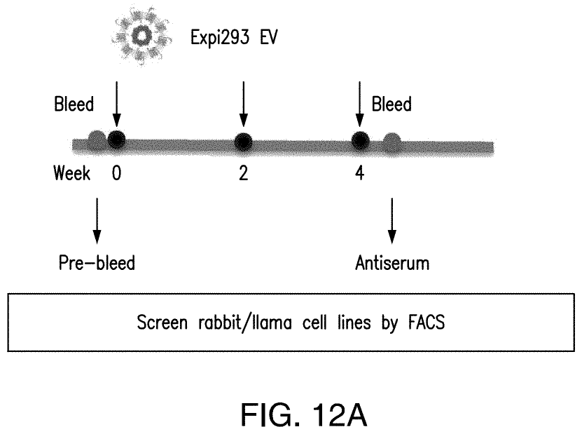

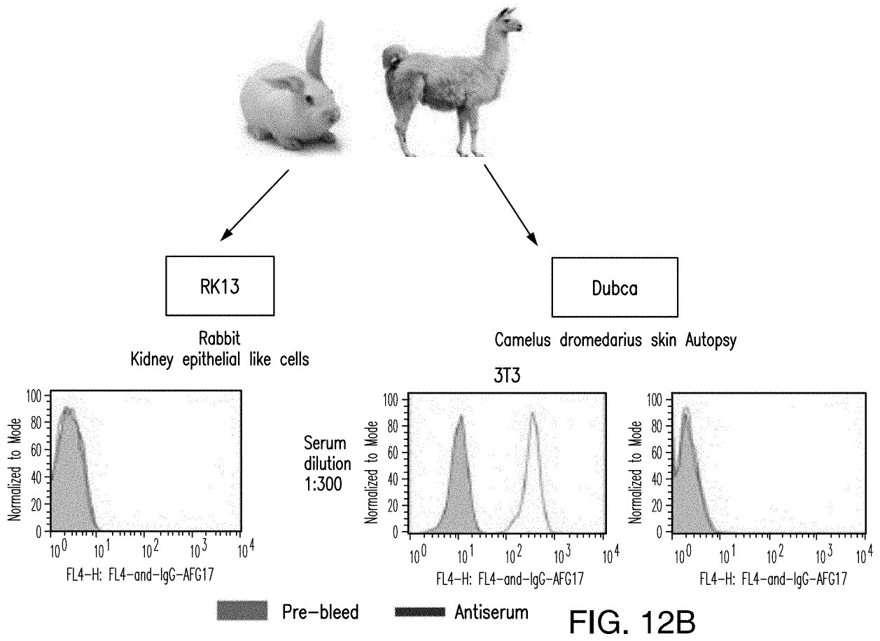

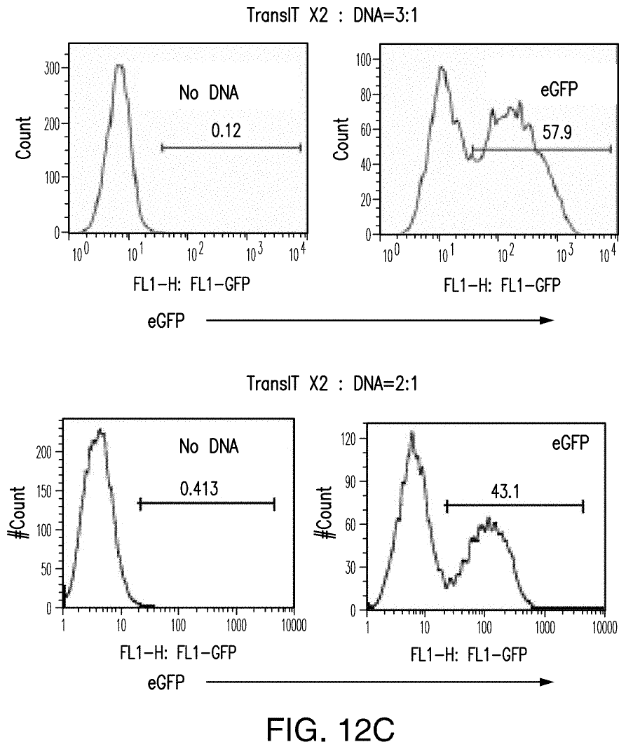

The present study screened rabbit, llama/camel, rat, and mouse cell lines for their transfection efficiency and their binding ability to EV immunized rat or mouse antiserum. SD rats, rabbits, and llamas and mice were immunized with Expi293F™ produced EVs on Week 0, Week 2 and Week 4. Serum samples were collected from the animals before (pre-bleed sample) and after immunization (antiserum sample) (FIG. 11A, FIG. 12A). The binding of the collected pre-bleed and antiserum samples to different cell lines, including the Expi293F™ cell line, RK13 cell line, Dubca cell line, RBA cell line and 3T3 cell line, were measured by FACS. Collected rat antiserum bound highly to 293 cell line, but not to RBA cell line which is a mammary gland adenocarcinoma cell line originated from SD rat. Collected mouse antiserum bound highly to RBA cell line, but not to 3T3 cell line, which is an embryonic fibroblast cell line derived from Balb/c mice (FIG. 11B). RBA cells were highly transfectable (FIG. 11C), and 3T3 cells were also reasonably transfectable (FIG. 11D). Collected rabbit antiserum did not bind to the RK13 cells and collected llama antiserum did not bind to the Dubca cells, but did bind to 3T3 cells (FIG. 12B). RK13 cells and Dubca cells were also transfectable (FIG. 12C). As such, the RBA cell line and 3T3 cell line can be used for screening antibodies from Expi293F™ EV immunized SD rats and mice, and the RK13 cell line and Dubca cell line can be used for screening antibodies from Expi293F™ EV immunized rabbits and llamas.

### Example 5: Developing Functional Monoclonal Antibodies Against Challenging Membrane Proteins with EV Antigens

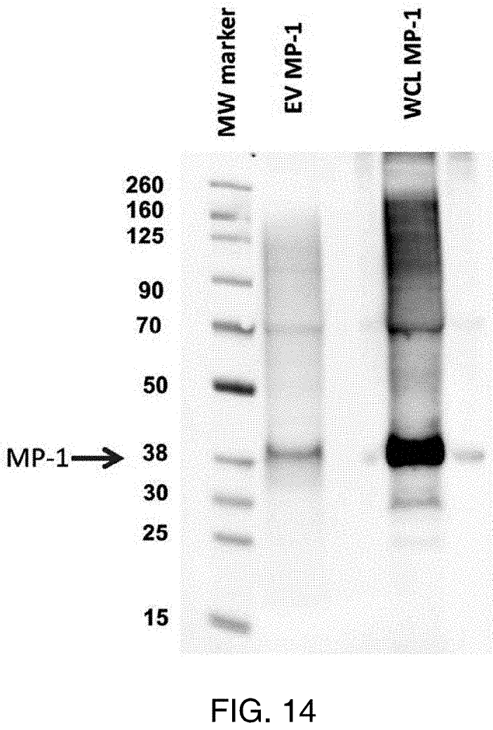

The Expi293F™ cell line was co-transfected with vesicle factor MLGag and membrane protein-1 (MP-1, a multi-pass membrane protein) constructs for 4 days, and EVs were collected from the culture media. Western blot confirmed that MP-1 was present in EVs and whole cell lysate (FIG. 14).

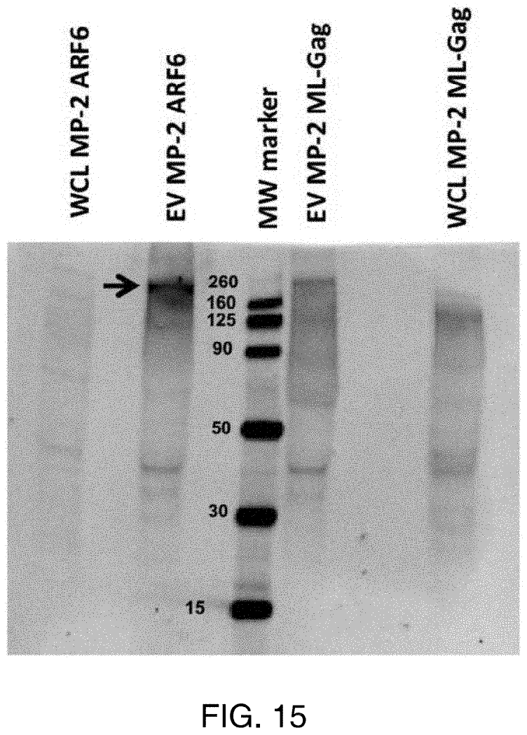

The Expi293F™ cell line was also co-transfected with vesicle factor MLGag or ARF6, and membrane protein-2 (MP-2, a multi-pass membrane protein that does not have intracellular domains comprising more than 110 amino acids) constructs for 4 days. Western blot confirmed that MP-2 was present in EVs and whole cell lysate (FIG. 15).

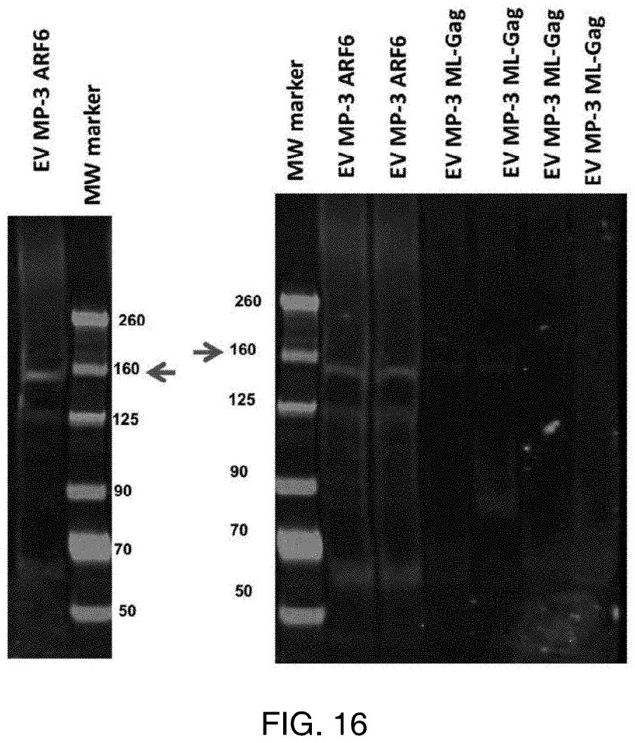

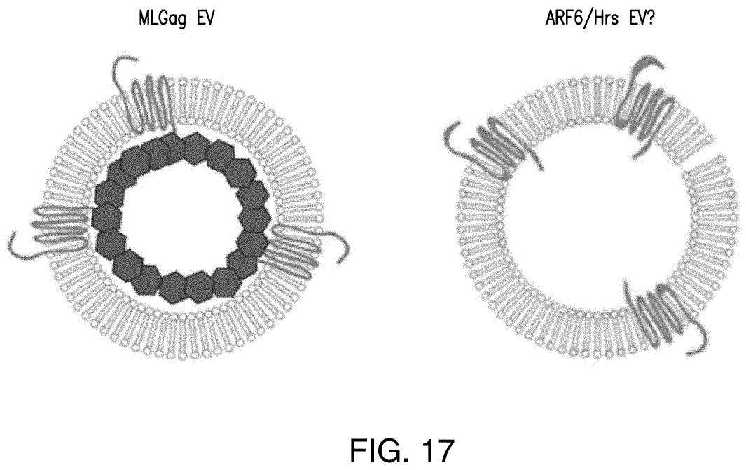

The Expi293F™ cell line was also co-transfected with vesicle factor MLGag or ARF6, and membrane protein-3 (MP-3, a multi-pass membrane protein that has an intracellular domain comprising more than 700 amino acids) constructs for 4 days. Western blot confirmed that MP-3 was present in ARF6 possessing EVs produced from cells transfected with ARF6, but not MLGag (FIG. 16). Without being limited to particular theory, the reason for such results could be that Gag capsid sterically blocks the incorporation of MPs with large intracellular domains (FIG. 17).

### Example 6: EV-Based ELISA to Screen Primary Antibodies Against Antigens in Native Format

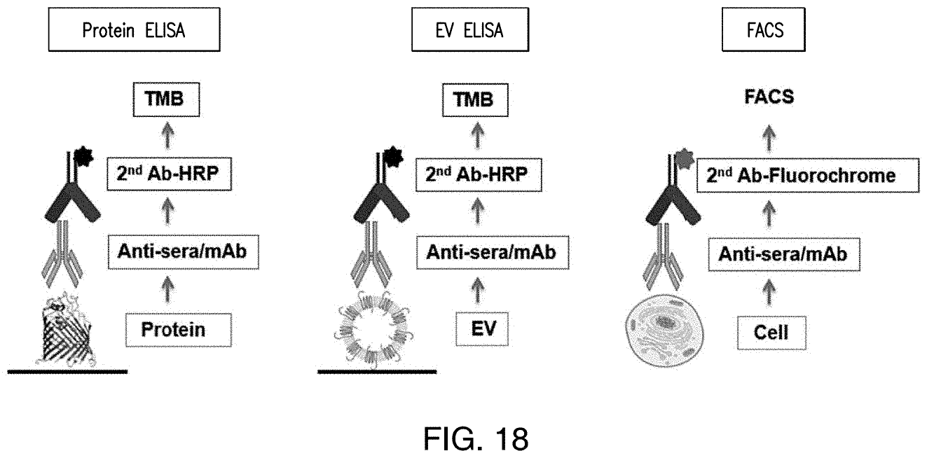

The present study compared the methods of using EV-based ELISA with cell-based FACS to screen primary antibodies against membrane proteins in their native forms. The working mechanisms of protein ELISA, EV-based ELISA and FACS were shown in FIG. 18.

Two membrane proteins, MP-4 and MP-5, were used as binding antigens for the present study. MP-4 is a single-pass membrane protein, and MP-5 is a multi-pass membrane protein.

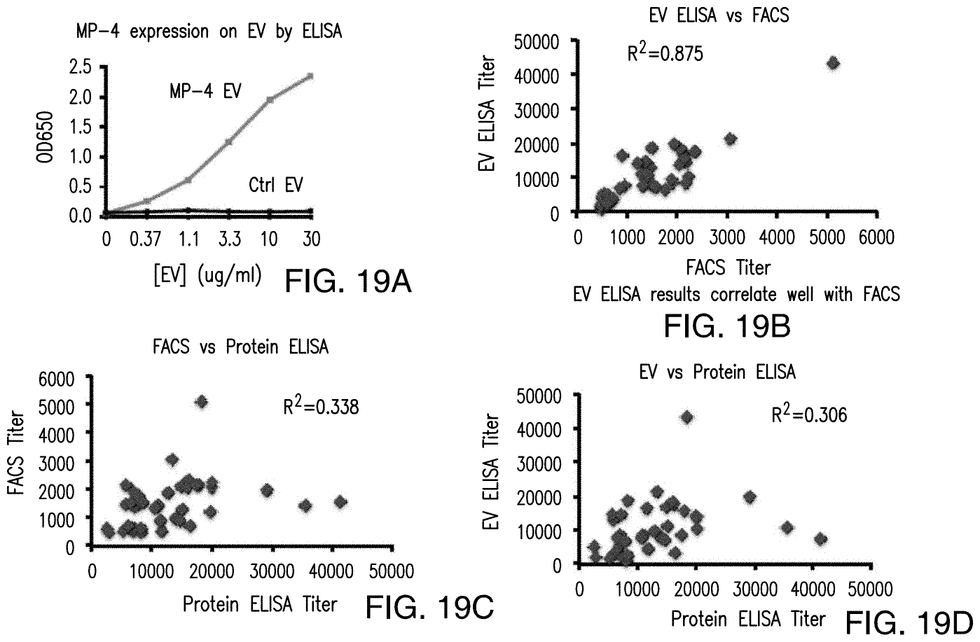

Anti-MP-4 hybridomas were generated from mice immunized with MP-4 using protein immunization. EVs comprising membrane-bound MP-4 were generated using MLGag for the EV-based ELISA. FIGS. 19A-19D showed that EV-based ELISA titer correlated well with FACS titer for MP-4, and EV-based ELISA results were consistent with the FACS results. In contrast, correlation between protein-based ELISAs with either EV-based ELISA or FACS was quite poor.

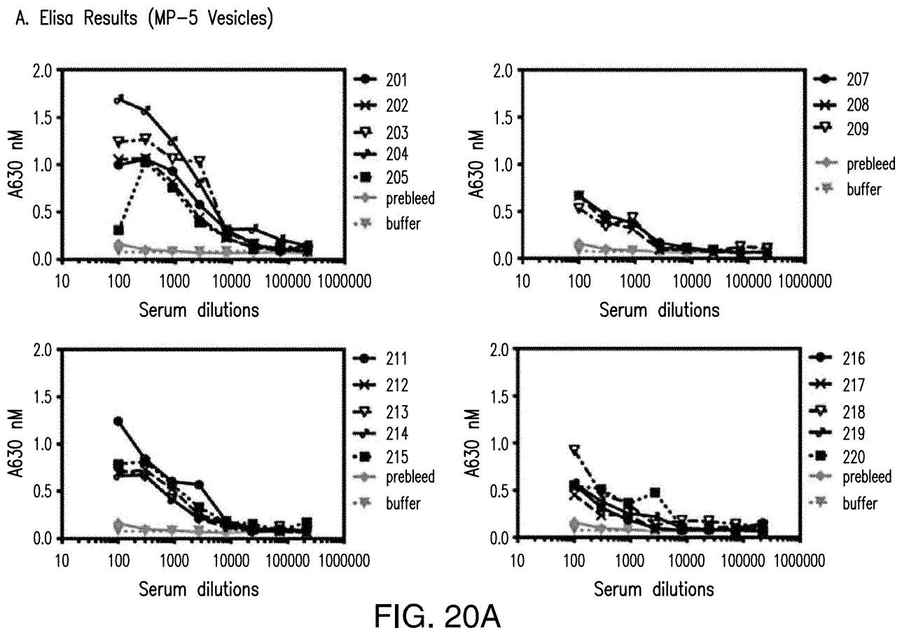

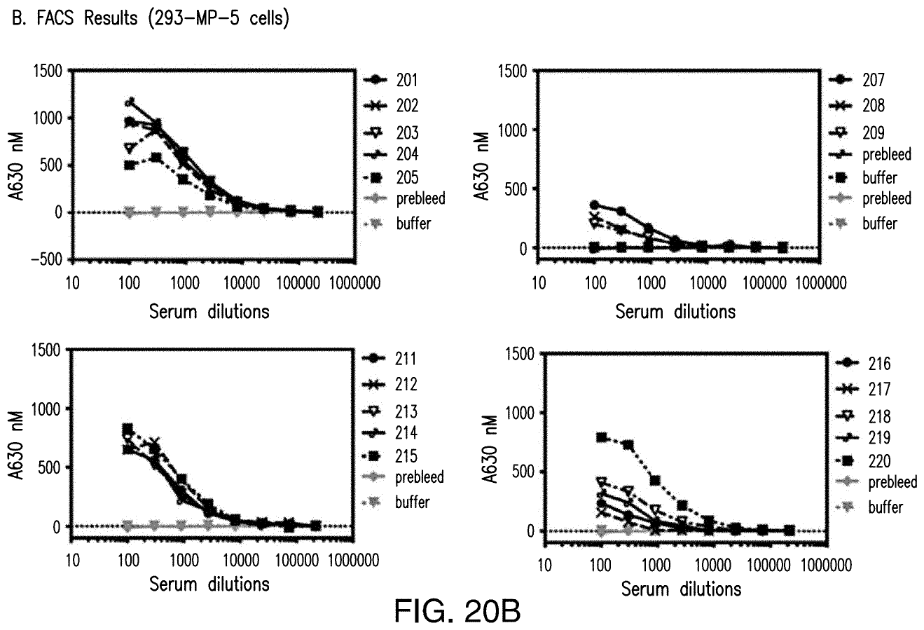

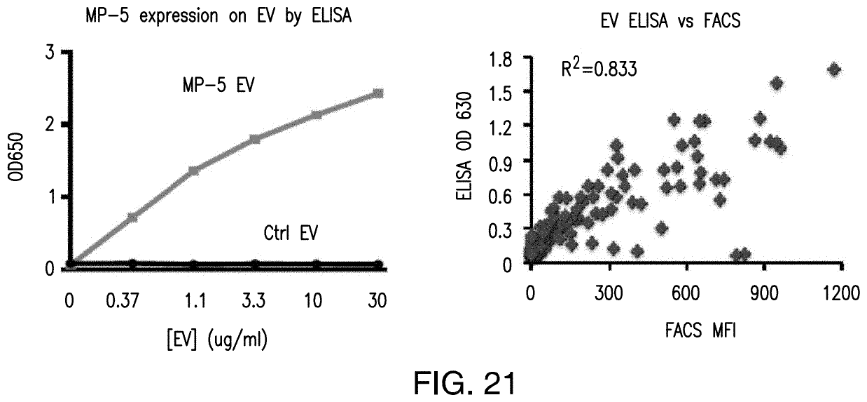

Anti-MP-5 sera and hybridomas were generated from mice immunized with MP-5 using DNA immunization. Similarly, EV-based ELISA correlated well with FACS (FIGS. 20A-20B, 21).

Therefore, the present study showed that EVs enabled ELISA-based detection of antibodies against complex membrane proteins, and EV-based ELISA can be used for screening primary antibodies against native format antigens.

### Example 7: Use of Antigen Expressing EVs for Monoclonal Antibody Discovery Against Challenging Antigen MP-6

MP-6 is a high value antibody-drug-conjugate (ADC) target for multiple cancers and is a challenging target for developing anti-MP-6 antibodies. EVs comprising membrane-bound MP-6 were produced by co-transfecting 293S cells with a vesicle factor of MLGag, Acyl.Hrs, ARF6, or ARRDC1, and MP-6 constructs. EVs were isolated by ultracentrifugation. The yields of EVs were shown in Table 2. Relative levels of MP-6 were measured by Western blot.

TABLE 2
Yields of EV in the cells transfected with each vesicle factor

| | Yield (mg) | Rel. MP-6 level |
|---|---|---|
| MLGag | 5.9 | 1.27 |
| Acyl.Hrs | 4.7 | 1 |
| ARF6 | 4.46 | 0.58 |
| ARRDC1 | 3.33 | 1.92 |

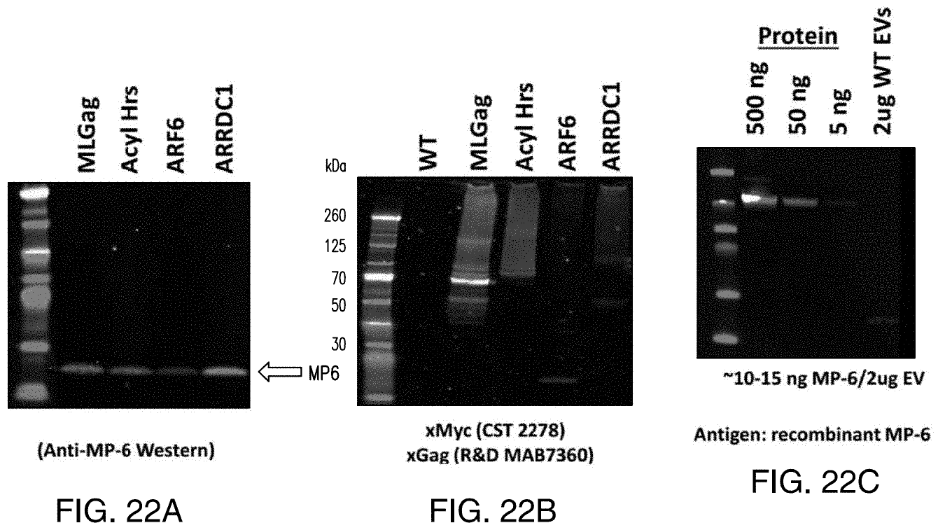

Analysis of initial batch of MP-6 EVs showed that MP-6 was expressed in the isolated EVs (FIG. 22A). Additionally, Western blot analysis confirmed that each of vesicle factors was also incorporated into the vesicle (FIG. 22B). A quantitative Western blot using a recombinant protein standard was used to measure the absolute amount of MP-6 incorporated in the vesicles (FIG. 22C).

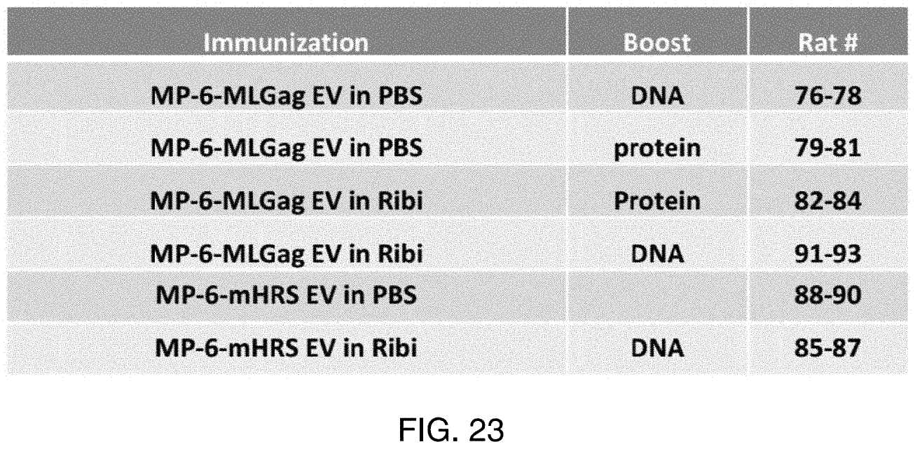

SD rats were immunized with the produced EVs along with a DNA or protein boost. EVs for immunization were prepared in PBS or Ribi (adjuvant). The immunization protocols were shown in FIG. 23. Antiserum were collected from the rats before or after the DNA or protein boost. Antibodies were purified from the collected antiserum, with a final concentration of 250, 50, 10, or 2 μg/ml. The levels of anti-MP-6 antibody in the purified antibodies were measured by FACS or Western Blot. RBA cells were transfected with or without MP-6 expressing construct using Lipofectamine 3000 (Lipofectamine:DNA=3:1) for 2 days. These RBA cells were used in FACS analysis.

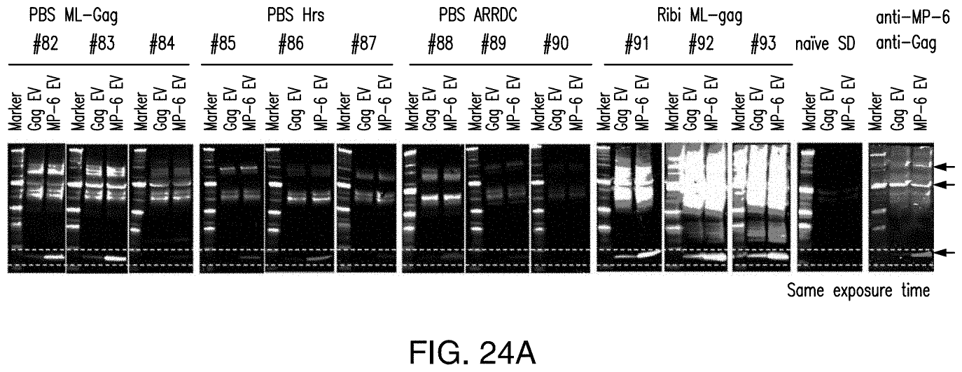

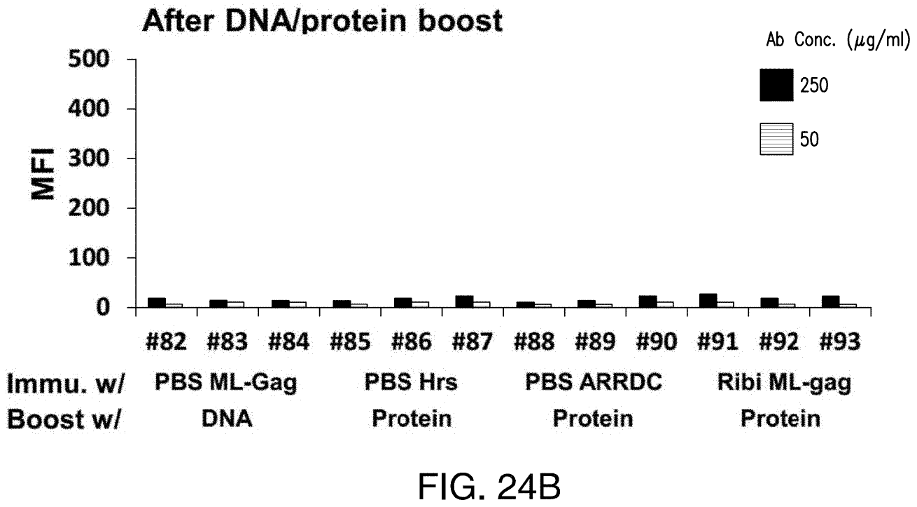

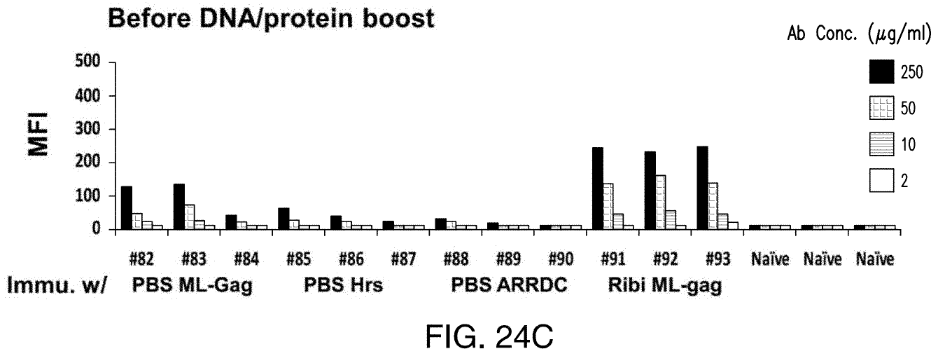

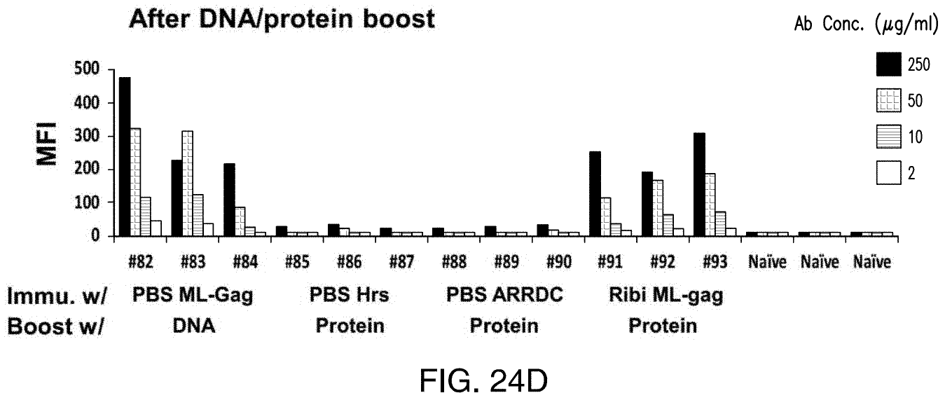

Western blot showed that the levels of anti-MP-6 antibody and anti-Gag antibody in the antiserum collected from the rats after the DNA or protein boost (FIG. 24A). It was found that antiserum collected from the rats after the DNA or protein boost had no significant binding to RBA cells (FIG. 24B). The present study also measured the antibody levels in the collected antiserum using RBA based FACS. The FACS results correlated well with western blot data (FIGS. 24C and 24D). As such, it showed that immunized primary antibodies did not show background binding to RBA cells, and IgG of the naïve SD rats did not bind to RBA transfected with MP-6. Only antibodies collected from rats immunized with MP-6 expressing EVs showed binding to RBA transfected with MP-6. As such, RBA-based FACS can be used for screening MP-6 antibodies produced by EV immunized rats.

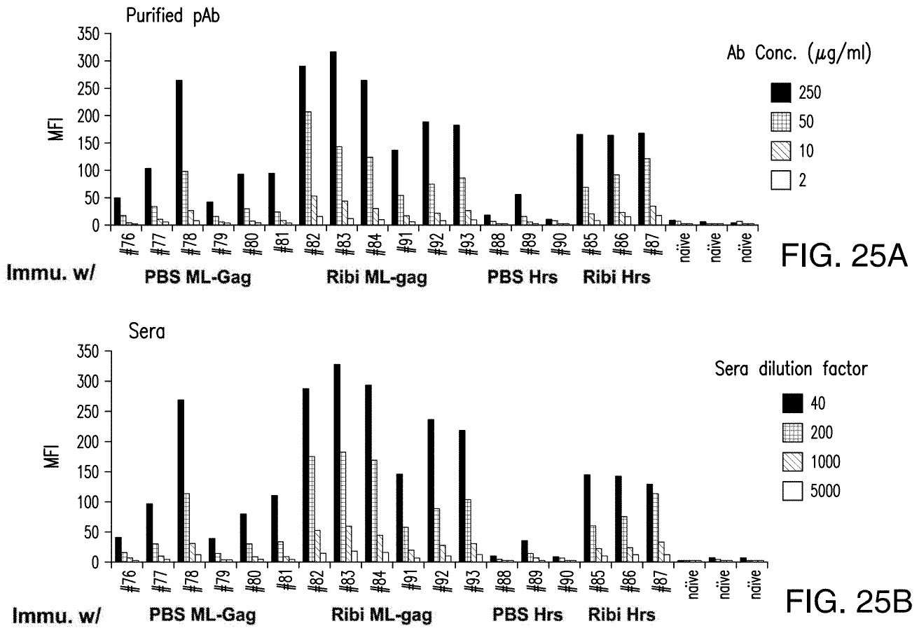

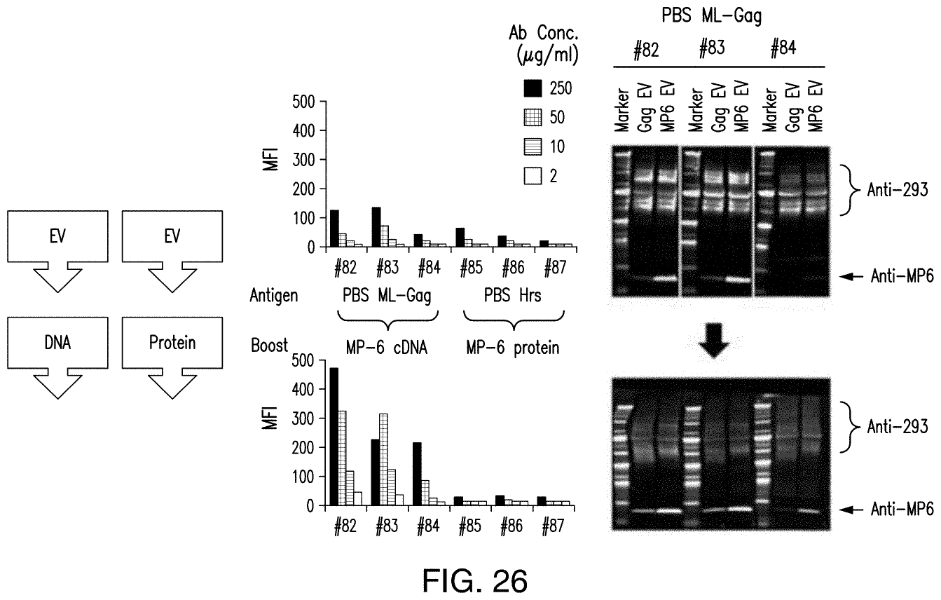

The present study found that DNA boost but not protein boost increased anti-MP-6 antibody titer in rats immunized with MP-6 expressing EVs (FIGS. 24A-24D, 26). Additionally, incorporating an adjuvant (Ribi) in the immunization process also increased anti-MP-6 antibody titer (FIGS. 24A-24D, 25A-25B).

The study showed that Ribi as an adjuvant increased antibody titer, and Acyl.Hrs EVs generated weaker antibody response than MLGag EVs (FIG. 25A). FIG. 25B showed that serum was appropriate for antibody titer check by FACS, and IgG purification was not needed.

### Example 8: Use of Antigen Expressing EVs for Monoclonal Antibody Discovery Against MP-7

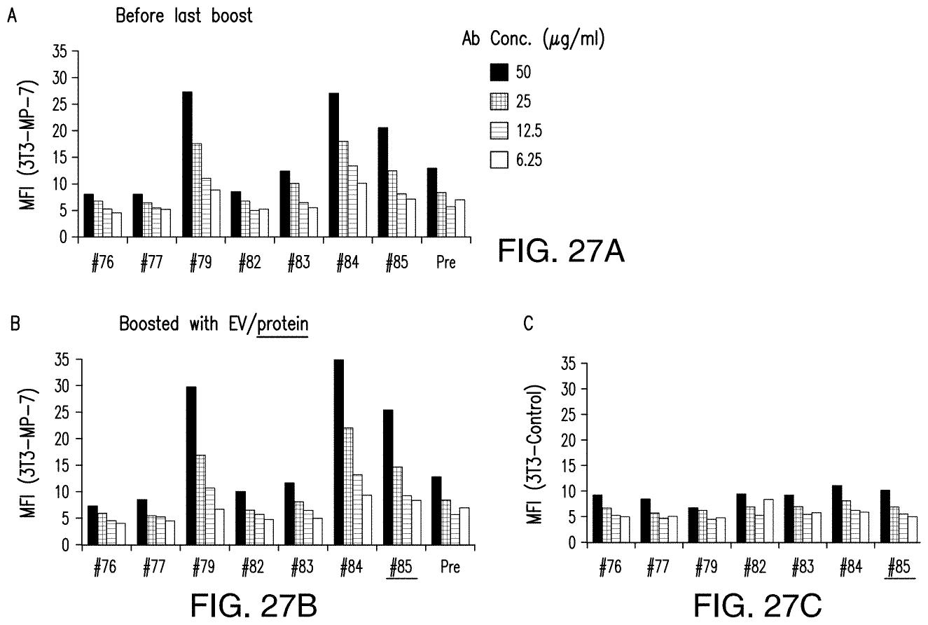

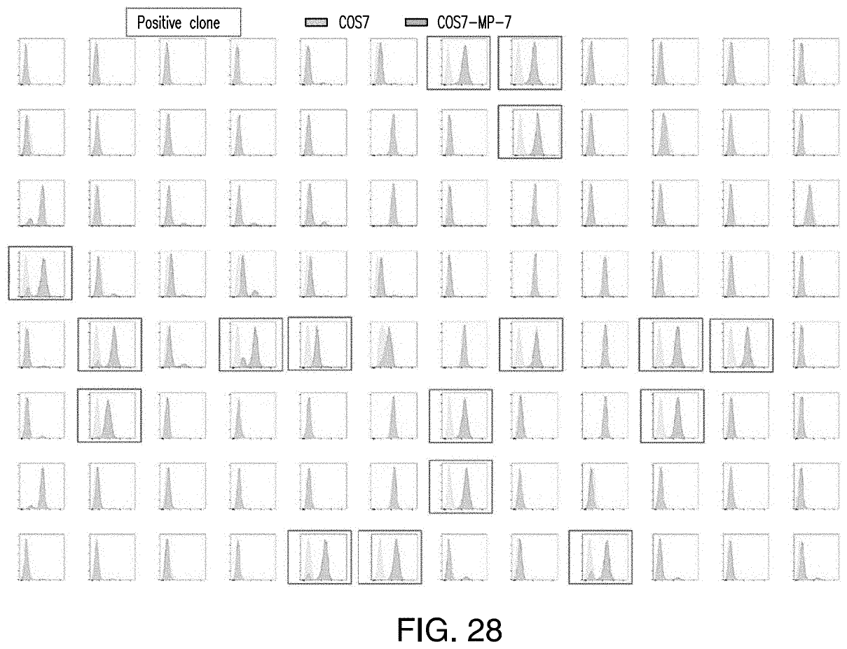

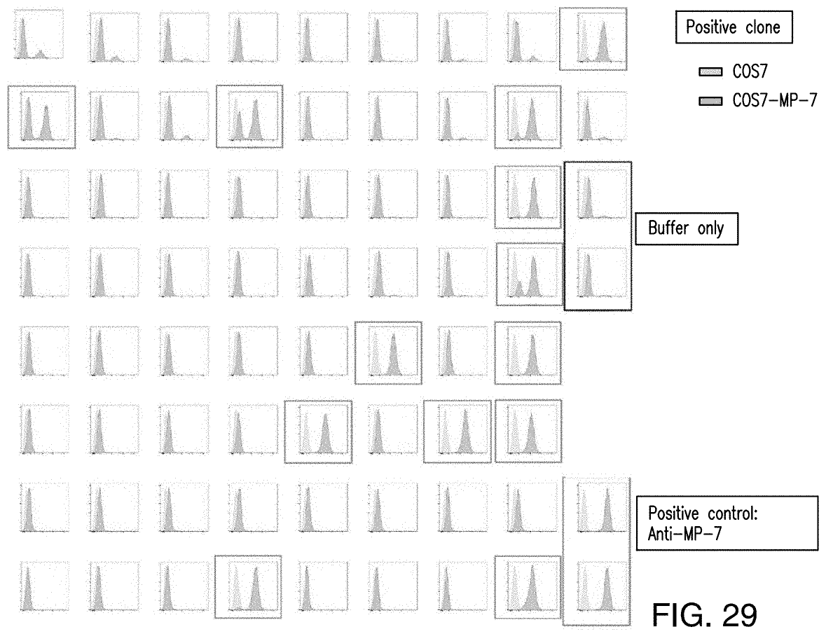

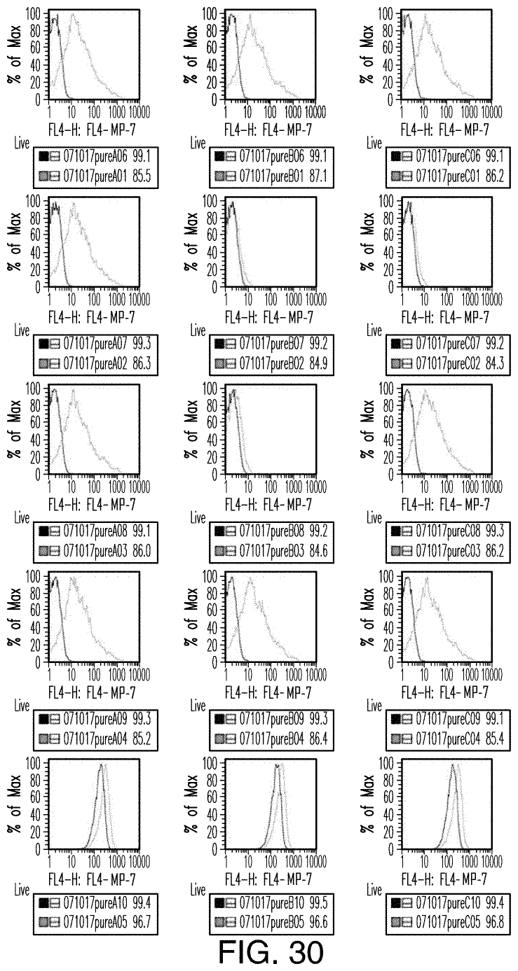

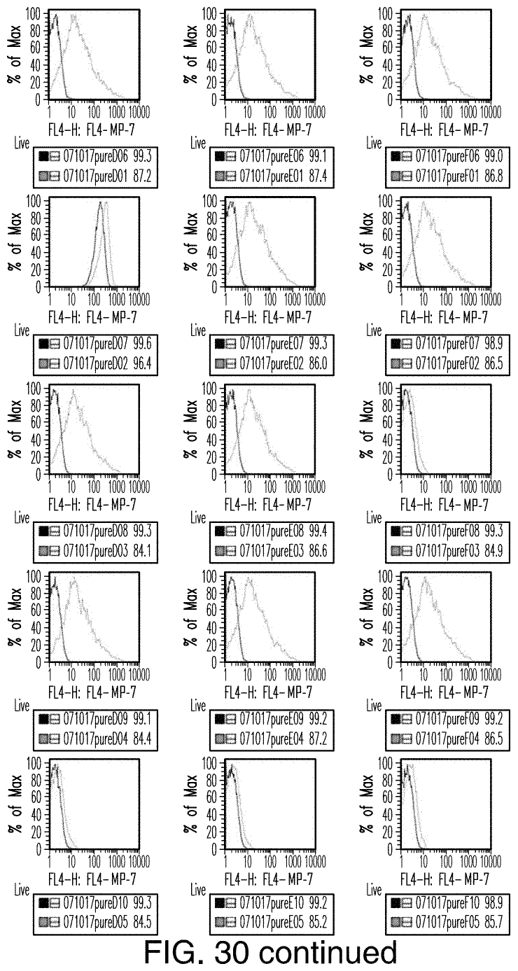

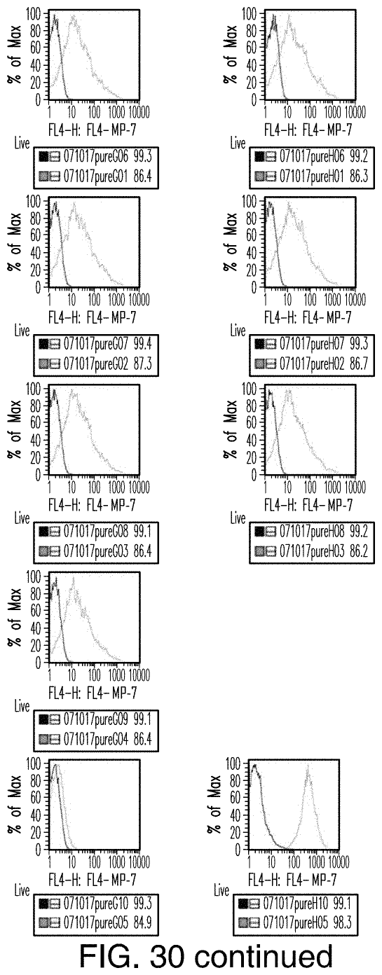

The present study used the immunization method developed in Example 7 to discover and generate monoclonal antibodies against membrane protein MP-7, a multi-pass membrane protein. Mouse anti-MP-7 primary antibodies were generated from knockout mice immunized with EVs comprising MP-7, and screened by FACS. Anti-MP-7 hybridomas were selected from the mice in which anti-serum showed significant binding to MP-7 expressing cells in FACS (FIGS. 27A-27C). The hybridomas were further screened by FACS to select anti-MP-7 antibody clones that showed strong binding to MP-7 in COST stable cells and endogenous cells (FIGS. 28-30).

### Example 9: Use of EV Comprising Antigen for Monoclonal Antibody Discovery Against MP-1

The present study used the immunization method developed in Example 7 to produce monoclonal antibodies against membrane protein MP-1. Rats were immunized with EVs comprising membrane bound MP-1 or MP-1 DNA, with a protein or DNA boost. Additional EVs were generated in which MP-1 was fused to 4 repeats of a universal T cell epitope from tetanus toxoid (MP-8 TCE4) (Demotz et al. J Immunol 1989; 142). The EVs were generated by expression of MLGag as the vesicle factor.

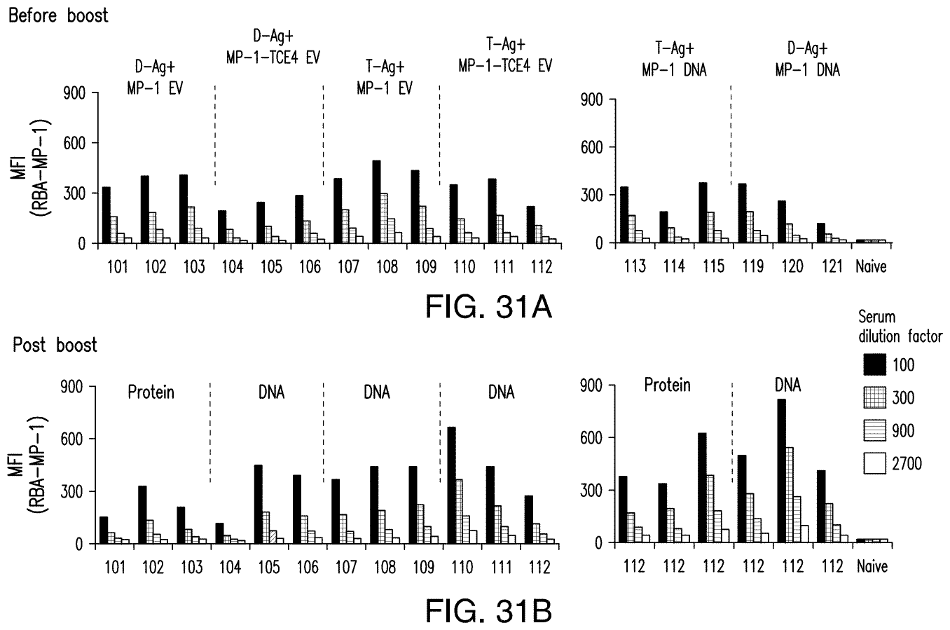

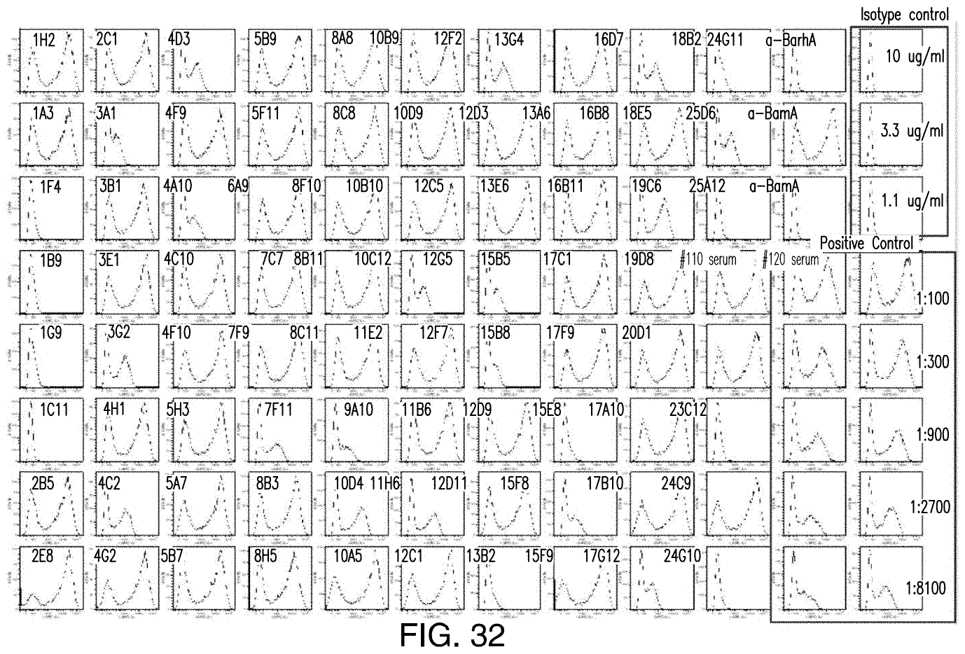

Antiserum collected from the rats was screened by FACS (FIGS. 31A-31B). It showed that DNA boost overall was more effective than protein boost in increasing antibody titer and that the addition of the T-cell epitope had no effect. Protein boost increased FACS titer in DNA immunized rats, suggesting some overlap of epitopes between the protein boost and the cell surface MP-1. Rats anti-MP-1 hybridomas were screened by FACS (FIG. 32). RBA cells were transfected with MP-1 DNA with Lipofectamine 3000 for 1 day, then stained with rat anti-MP-1 hybridoma supernatants followed by AF647-anti-rat IgG.

### Example 10: Use of EVs Comprising Membrane-Bound Antigen for Monoclonal Antibody Discovery Against MP-8

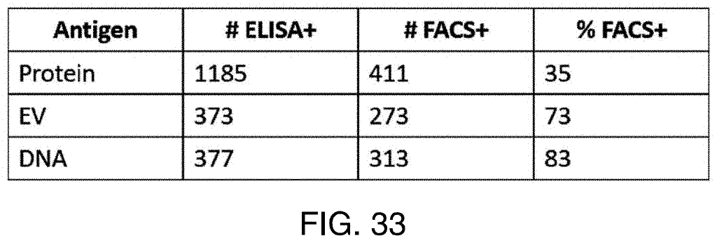

The present study used the immunization method developed in Example 7 to discover and generate monoclonal antibodies against membrane protein MP-8, a single pass membrane protein. Rats were immunized with only protein, DNA encoding for MP-8, or EVs comprising membrane-bound MP-8 (generated by using MLGag as the vesicle factor). ELISA and FACS results were shown in FIG. 33. The results show that while EV immunizations result in fewer ELISA+ clones compared to protein immunizations, the EV immunizations can generate a higher percentage of FACS+ antibodies compared to protein immunizations.

### Example 11: Use of Fluorescent EVs Comprising Membrane-Bound Antigen for Sorting of B Cells from Immunized Animals

The present study used the immunization method developed in Example 7 to discover and generate monoclonal antibodies against membrane protein MP-9, a multi-pass membrane protein. Rats and rabbits were immunized with EVs comprising MP-9 and MP-9 DNA. The EVs were generated by expression of MLGag as the vesicle factor.

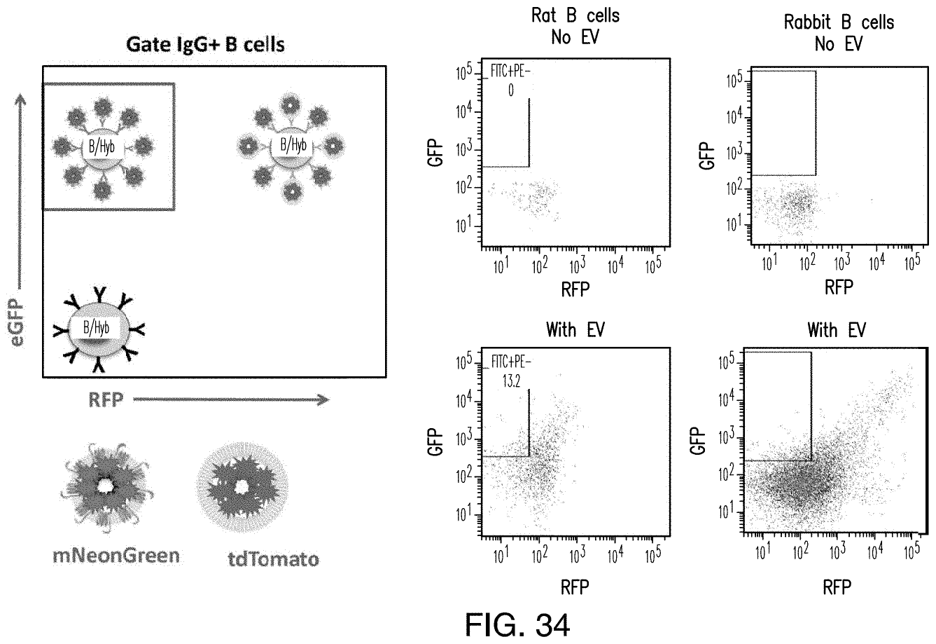

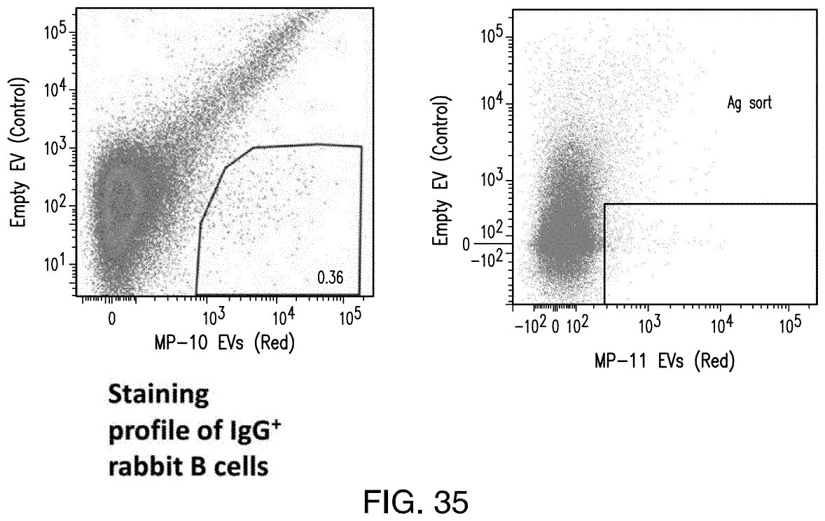

PBMCs were obtained from both rats and rabbits and stained with GFP-labeled MP-9 containing EVs and RFP-labeled EVs without MP-9. Staining of IgG+ B cells are shown in FIG. 34. The results show two populations of B cells that are stained with the EVs. The GFP/RFP+ population represents B cells that detect non-MP-9 proteins in the EV. The GFP-only labeled population, indicated in box, represents B cells that specifically detect MP-9. Using two other MPs (MP-10 and MP-11), it was shown that rabbit IgG+ B cells can be stained with RFP-labeled MP EVs and GFP-labeled empty EVs (FIG. 35). In each case, there is a clear population of RFP-only labeled B cells that specificity detect the MP.

### Example 12: Use of EV's to Generate Monoclonal Antibodies to Membrane Proteins of a Protein Complex

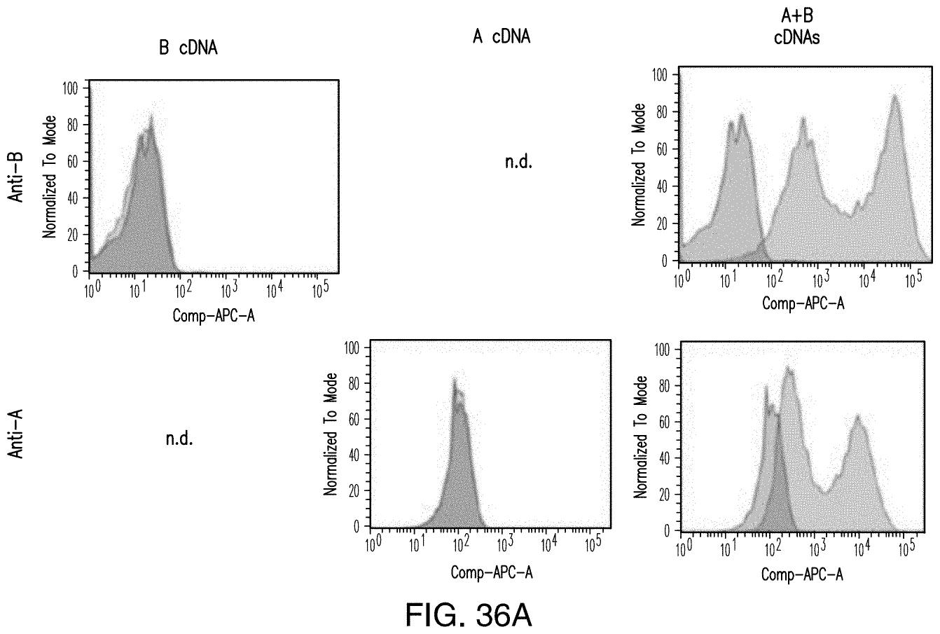

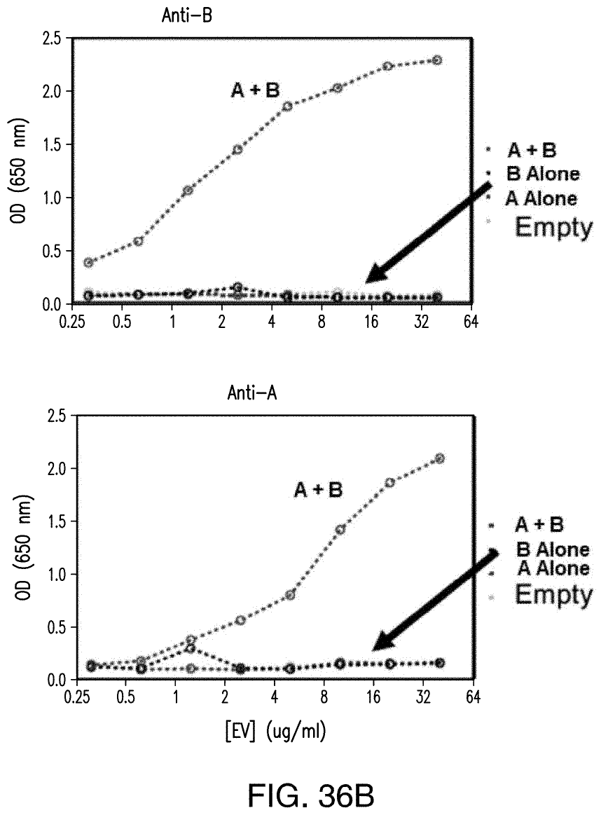

The present study used the immunization method developed in Example 7 to discover and generate monoclonal antibodies against membrane proteins within a protein complex. The protein complex comprises 6 different membrane proteins (MP-12, MP-13, 2 copies of MP-14, MP-15, MP-16 and 2 copies of MP-17). MP-12 and MP-13 dimerize to form a receptor (referred to herein as Receptor “A” in FIGS. 36A-B) and MP-14, MP-15, MP-16 and MP-17 form a complex (referred to herein as co-receptor “B” in FIGS. 36A-B) that functions as a co-receptor of the receptor formed by MP-12 and MP-13. Two polycistronic expression vectors were generated encoding for either both MP-12 and MP-13 or all four of MP-14, MP-15, MP-16 and MP-17. To confirm formation of the complex at the cell surface, Expi293 cells were transiently transfected with (i) MP-12 and MP-13-encoding cDNA, (ii) MP-14, MP-15, MP-16 and MP-17-encoding cDNA or both (i) and (ii). Expression of MP-14, MP-15, MP-16 and MP-17 and expression of MP-12 and MP-13 was detected by flow cytometry when all proteins were co-expressed, confirming assembly of the full complex (FIG. 36A). EVs were generated containing the full protein complex and incorporation was confirmed by ELISA (FIG. 36B). EVs were generated by expression of MLGag.

Rats were immunized with EVs comprising the complex of the 6 membrane proteins (MP-12, MP-13, MP-14, MP-15, MP-16 and MP-17). Subsequent characterization of monoclonal antibodies derived from the rats showed successful discovery of FACS+ antibodies that bound to proteins within the complex, e.g., either MP-12/MP-13, MP-14, MP-14/MP-16 or MP-14/MP-15 (Table 3). These data show that EVs can be used to generate antibodies against membrane proteins that are present in protein complexes.

TABLE 3
Identification of complex-specific binding antibodies

| | ELISA⁺ on MP- | FACS⁺ on cells that |
|---|---|---|
| | 14/MP-16 or MP-14/ | endogenously express |
| Specificity | MP-15 protein | the complex |
| MP-12/MP-13 | n.d. | >50 |
| MP-14 | 30 | 18 |
| MP-14/MP-16 | 20 | 1 |
| MP-14/MP-15 | 15 | 3 |

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the inventions of which are incorporated herein by reference in their entireties for all purposes.

What is claimed is:

1. A method for producing an antibody that specifically binds to a protein comprising:
(a) producing a plurality of extracellular vesicles (EVs) comprising a heterologous protein by (i) expressing the heterologous protein in a cell exposed to a vesicle factor, (ii) culturing the cell in a medium and (iii) isolating the plurality of EVs comprising the heterologous protein from the medium, wherein the vesicle factor is selected from the group consisting of Acyl.Hrs, ARRDC1, ARF6 and a combination thereof;
(b) immunizing an animal by administering the plurality of EVs to the animal; and
(c) isolating an antibody that binds to the heterologous protein from the animal.

2. The method of claim 1, wherein the cell is a non-adherent cell.

3. A method for producing an antibody that specifically binds to a protein comprising:
(a) producing a plurality of extracellular vesicles (EVs) comprising a heterologous protein by (i) expressing the heterologous protein in a cell, (ii) culturing the cell in a medium and (iii) isolating the plurality of EVs comprising the heterologous protein from the medium, wherein the cell is a non-adherent cell;
(b) immunizing an animal by administering the plurality of EVs to the animal; and
(c) isolating an antibody that binds to the heterologous protein from the animal.

4. The method of claim 3, wherein producing the plurality of EVs further comprises expressing a vesicle factor in the cell.

5. The method of claim 4, wherein the vesicle factor is selected from the group consisting of MLGag, Acyl.Hrs, ARRDC1, ARF6 and a combination thereof.

6. The method of claim 1, wherein the heterologous protein is a membrane protein.

7. The method of claim 6, wherein: (i) the membrane protein is a single-pass membrane protein; (ii) the membrane protein is a multi-pass membrane protein; (iii) the membrane protein is a member of a protein complex; and/or (iv) the membrane protein is not a transmembrane protein but is a protein that is part of a complex with a transmembrane protein.

8. The method of claim 1, further comprising (i) administering an adjuvant to the animal concurrently with the EVs and/or (ii) a boost to the animal to enhance an immune response in the animal to the protein.

9. An isolated antibody or an antigen-binding portion thereof produced by the method of claim 1.

10. An isolated nucleic acid encoding the antibody or antigen-binding portion thereof of claim 9.

11. A host cell comprising the nucleic acid of claim 10.

12. A method of producing an antibody or antigen-binding portion thereof comprising culturing the host cell of claim 11 under conditions suitable for expression of the antibody.

13. A pharmaceutical composition comprising the isolated antibody or antigen-binding portion thereof of claim 9 and a pharmaceutically acceptable carrier.

14. A method of treating an individual having a disease comprising administering to the individual an effective amount of the isolated antibody or antigen-binding portion thereof of claim 9 or the pharmaceutical composition of claim 13.

15. A method for detecting an antibody in a sample comprising:
(a) incubating a sample with a capture reagent, wherein the capture reagent comprises a plurality of EVs comprising membrane-bound antigen, and the antibody binds specifically to the membrane-bound antigen; and
(b) contacting the antibody binding to the capture reagent with a detectable antibody to detect the bound antibody, wherein the detectable antibody binds specifically to the antibody,
wherein the plurality of EVs are generated by (i) expression of the membrane-bound antigen in a cell, (ii) culturing the cell in vitro in a medium to produce the plurality of EVs displaying the membrane-bound antigen and (iii) isolating the plurality of EVs displaying the membrane-bound antigen from the medium, and
wherein the cell is exposed to a vesicle factor selected from the group consisting of Acyl.Hrs, ARRDC1, ARF6 and a combination thereof and/or the cell is a non-adherent cell.

16. The method of claim 15 further comprising (c) measuring the amount of the antibody detected in (b), wherein the amount is quantitated using a standard curve.

17. A method for sorting antibody-producing cells comprising:
(a) incubating the antibody-producing cells with a plurality of EVs wherein the plurality of EVs comprise:
i. a first population of EVs comprising a membrane-bound antigen and a first detectable marker, wherein a subset of the antibody-producing cells bind specifically to the membrane-bound antigen; and
ii. a second population of EVs lacking the membrane-bound antigen but comprising a second detectable marker distinguishable from the first marker; and
(b) sorting the antibody-producing cells based on their binding to either the first population of EVs or to a combination of the first population of EVs and the second population of EVs,
wherein the first population of EVs is generated by (i) expression of the membrane-bound antigen and the first detectable marker in a first cell, (ii) culturing the first cell in vitro in a medium to produce the plurality of EVs displaying the membrane-bound antigen and (iii) isolating the plurality of EVs displaying the membrane-bound antigen from the medium,
wherein the second population of EVs is generated by (i) expression of the second detectable marker in a second cell, (ii) culturing the second cell in vitro in a medium to produce the plurality of EVs comprising the second detectable marker and (iii) isolating the plurality of EVs displaying the second detectable marker from the medium, and
wherein (i) the first cell and/or the second cell is exposed to a vesicle factor selected from the group consisting of Acyl.Hrs, ARRDC1, ARF6 and a combination thereof and/or (ii) the first and/or second cell is a non-adherent cell.

18. The method of claim 17, wherein the sorting is performed by fluorescence-activated cell sorting.

19. A method for producing a plurality of extracellular vesicles (EVs) displaying a protein, comprising:
(a) expressing a heterologous protein in a cell;
(b) culturing the cell in a medium; and
(c) isolating the plurality of EVs comprising the heterologous protein from the medium,
wherein the cell is exposed to a vesicle factor selected from the group consisting of Acyl.Hrs, ARRDC1, ARF6 and a combination thereof and/or the cell is a non-adherent cell.

20. A kit for detecting an antibody in a sample comprising:
(a) a capture reagent that comprises a plurality of EVs comprising membrane-bound antigen, wherein the antibody to be detected binds specifically to the antigen; and
(b) a detectable antibody that binds specifically to the antibody to be detected,
wherein the plurality of EVs are generated by (i) expression of the membrane-bound antigen in a cell, (ii) culturing the cell in vitro in a medium to produce the plurality of EVs displaying the membrane-bound antigen and (iii) isolating the plurality of EVs displaying the membrane-bound antigen from the medium, and
wherein the cell is exposed to a vesicle factor selected from the group consisting of Acyl.Hrs, ARRDC1, ARF6 and a combination thereof and/or the cell is a non-adherent cell.
